(19) United States
(12) Patent Application Publication
(10) Pub. No.: US 2026/0207947 A1
(43) Pub. Date: Jul. 23, 2026

(54) TIME OFFSET UPDATE FOR IMPLANTABLE MEDICAL DEVICES

(71) Applicant: Medtronic, Inc., Minneapolis, MN (US)

(72) Inventors: Sakethram Karumuri, Maple Grove, MN (US); Jeffrey R. Glass, Dayton, MN (US); Kunal J. Paralikar, Roseville, MN (US); Christopher M. Georgen, Whitefish Bay, WI (US)

(21) Appl. No.: 19/455,394

(22) Filed: Jan. 21, 2026

Related U.S. Application Data
(60) Provisional application No. 63/748,829, filed on Jan. 23, 2025.

(51) Int. Cl. A61N 1/372

(52) U.S. Cl. CPC A61N 1/37235

(57) ABSTRACT

Systems, devices, and techniques are described for updating time for an implantable medical device (IMD). An example system includes processing circuitry of the IMD, where the processing circuitry is configured to determine time information for the IMD based on an internal clock of the IMD and a reference time stored in a memory of the IMD. The processing circuitry is configured to control telemetry circuitry to transmit the time information to an external device. The processing circuitry is configured to receive, from the external device, an offset to the time information. The processing circuitry is configured to store the offset in the memory of the IMD. The processing circuitry is configured to control the IMD to deliver therapy according to the time information, the offset, and a therapy program that at least partially defines the therapy.

### CROSS-REFERENCE TO RELATED APPLICATION

This application claims the benefit of and priority to U.S. Provisional Patent Application No. 63/748,829 filed January 23, 2025, the entire disclosure of which is incorporated by reference herein.

### TECHNICAL FIELD

This disclosure relates to implantable medical devices, and, more specifically, managing time of implantable medical devices.

### BACKGROUND

Medical devices may be external or implanted and may be used to monitor patient signals such as cardiac activity, biological impedance and to deliver electrical stimulation therapy to patients via various tissue sites to treat a variety of symptoms or conditions such as chronic pain, tremor, Parkinson’s disease, epilepsy, urinary or fecal incontinence, sexual dysfunction, obesity, or gastroparesis and other conditions. In some examples, a medical device may include timekeeping circuitry that is used to deliver therapy according to a therapy schedule.

### SUMMARY

In general, this disclosure is directed to devices, systems, and techniques for determining and updating an offset to time information for an implantable medical device (IMD). This disclosure is generally directed to devices, systems, and techniques for determining time information for an IMD based on an internal clock and reference time stored in memory of the IMD, determining an offset to the time information to account for differences between the time information of the IMD and local time at initialization (e.g., programming by a clinician programmer), and subsequently updating the offset to account for changes to calendar time, such as a daylight savings transition and/or change of time zone (e.g., due to travel). Instead of relying on actual time updates, wherein timekeeping circuitry or other timekeeping functionality is periodically recalibrated to match an external source (e.g., time information from server), the system can save power and avoid unreliable time sources by maintaining internal timekeeping configurations and applying an offset that can be adjusted (e.g., increasing/decreasing the offset by an integer number of time zone offset intervals, such as hours or fractions thereof (e.g., 15 minute or 30 minute intervals)) to reflect a change in local time due to travel (i.e., change of time zone) and/or transition from standard time to daylight savings time (DST) or vice versa.

In one example, a system includes processing circuitry of an IMD. The processing circuitry is configured to determine time information for the IMD based on an internal clock of the IMD and a reference time stored in a memory of the IMD. The processing circuitry is configured to control telemetry circuitry to transmit the time information to an external device. The processing circuitry is configured to receive, from the external device, an offset to the time information. The processing circuitry is configured to store the offset in the memory of the IMD. The processing circuitry is configured to control the IMD to deliver therapy according to the time information, the offset, and a therapy program that at least partially defines the therapy.

In another example, a method includes determining, by processing circuitry, time information for an IMD based on an internal clock of the IMD and a reference time stored in a memory of the IMD. The method includes controlling, by the processing circuitry, telemetry circuitry to transmit the time information to an external device. The method includes receiving, by the processing circuitry, from the external device, an offset to the time information. The method includes storing, by the processing circuitry, the offset in the memory of the IMD. The method includes controlling, by the processing circuitry, the IMD to deliver therapy according to the time information, the offset, and a therapy program that at least partially defines the therapy.

In another example, a system includes an external device and an IMD. The IMD includes processing circuitry configured to determine time information for the IMD based on an internal clock of the IMD and a reference time stored in a memory of the IMD. The processing circuitry is configured to control telemetry circuitry to transmit the time information and the offset to the external device. The processing circuitry is configured to receive, from the external device, an offset to the time information. The processing circuitry is configured to store the offset in the memory of the IMD. The processing circuitry is configured to control the IMD to deliver therapy according to the time information, the offset, and a therapy program that at least partially defines the therapy.

The details of one or more examples of this disclosure are set forth in the accompanying drawings and the description below. Other features, objects, and advantages of this disclosure will be apparent from the description and drawings, and from the claims.

### BRIEF DESCRIPTION OF DRAWINGS

FIG. 1 is a conceptual diagram illustrating an example medical device system including an including an implantable medical device, which may be a leadless neurostimulation device, implanted near a tibial nerve in a leg of a patient.

FIG. 2 is a block diagram illustrating example components of the implantable medical device of FIG. 1.

FIG. 3 is a block diagram of an example programmer.

FIG. 4 is a block diagram of an example external charging device.

FIG. 5 is a flow chart illustrating an example technique for setting time information and an offset to time information for an implantable medical device.

FIG. 6 is a flow chart illustrating an example technique for updating an offset to time information for an implantable medical device.

FIG. 7 is a flow chart illustrating an example technique for updating an offset to time information for an implantable medical device.

FIG. 8 is a flow chart illustrating an example technique for toggling on or off time offset adjustments for an implantable medical device.

FIG. 9 is a flow chart illustrating an example technique for updating an offset to time information for an implantable medical device.

FIG. 10 is a flow chart illustrating an example technique for exchanging information between an implantable medical device and an external device.

FIG. 11 is a flow chart illustrating an example technique for controlling an implantable medical device to deliver therapy according to a therapy program.

FIG. 12 is a flow chart illustrating an example technique for updating an offset to time information for an implantable medical device.

FIG. 13 is a flow chart illustrating an example technique for updating an offset to time information for an implantable medical device.

FIG. 14 is a flow chart illustrating an example technique for controlling an implantable medical device to deliver therapy according to a therapy program.

FIG. 15 is a flow chart illustrating an example technique for communicating maintenance reminders regarding an implantable medical device.

Like reference characters denote like elements throughout the description and figures.

### DETAILED DESCRIPTION

This disclosure describes devices, systems, and techniques for determining and updating an offset to time information for an implantable medical device (IMD). Scheduled therapy delivered by an IMD (e.g., neuromodulation delivered to a sacral nerve, pudendal nerve, tibial nerve, spinal cord, peripheral nerve, etc.) is an efficient and efficacious mode of treatment. To maintain efficacy, therapy may be delivered consistently at a certain time of the day and for a certain duration of time. In some examples, an IMD, such as an implantable neurostimulator (INS), may interact with a programmer (e.g., a clinician programmer (CP) and/or a patient programmer (PP)) to receive information regarding a therapy program that at least partially defines parameters of therapy to be delivered by the IMD.

In some examples, timekeeping functionality may consume relatively high power for an IMD or programmer device, often requiring connection with external devices, WiFi networks, cellular towers, satellites, or a combination thereof. As a result, providing timekeeping and correction capabilities to an IMD may be difficult and an undesirable use of resources because of battery size limitations for implantable devices. However, in examples provided herein, the IMD does not need to track true time or otherwise change or adjust an internal clock. Instead, the IMD can use an internal clock that counts seconds, or other units of time, that have passed since a reference time that is stored in memory of the IMD to manage its time-based therapy delivery. The IMD can deliver therapy at a scheduled time and duration according to the internal clock when the user remains in a given time-zone and is not impacted by daylight savings transitions. However, the IMD can obtain an offset using external interaction to maintain a given schedule even in case of time zone changes, daylight savings transitions, or other user needs to change the schedule.

In a patient home environment, the PP can potentially update the IMD time to enable schedule changes. However, the PP (e.g., a patient’s handset) may not be able to maintain time accuracy due to lack of WiFi connectivity and/or frequent phone battery discharge. Thus, there may be a need for a solution that reliably maintains the therapy schedule for a patient when they travel to a different time-zone, their time-zone undergoes a daylight savings transition, or the patient wants their therapy schedule to be changed to a new time.

Devices, systems, and techniques are described herein for determining time information for an IMD based on an internal clock and reference time stored in memory of the IMD, determining an offset to the time information to account for differences between the time information of the IMD and local time at initialization (e.g., factory programming or programming by a clinician programmer), and subsequently updating the offset to account for a daylight savings transition and/or change of time zone (e.g., due to travel). Instead of relying on actual time updates, wherein timekeeping circuitry (or any components of the IMD) is periodically recalibrated to match an external source (e.g., time information from server), the system can save power and avoid unreliable sources by maintaining internal timekeeping configurations and making limited adjustments to the time using an offset (e.g., increasing/decreasing the offset by an integer number of time zone offset intervals, such as hours or fractions thereof) to reflect a change in local time due to travel (i.e., change of time zone) and/or transition from standard time to daylight savings time (DST) or vice versa.

Although the devices, systems, and techniques described herein are described primarily in the context of IMDs configured to provide tibial nerve stimulation, the techniques described herein may be applicable to other devices configured for other types of therapy. For example, the techniques of this disclosure may be applicable for other types of devices configured for invasive or noninvasive neuromodulation for pain relief, muscle activation, and/or other therapeutic benefits such as, but not limited to, deep brain stimulation (DBS), spinal cord stimulation (SCS), sacral nerve stimulation (SNS), cardiac stimulation, pacing, defibrillation, or other cardiac therapy, peripheral nerve stimulation or therapy, drug delivery (e.g., via a drug pump), circulatory support (e.g., mechanical circulatory support), or any other device (e.g., medical device) that includes timekeeping circuitry.

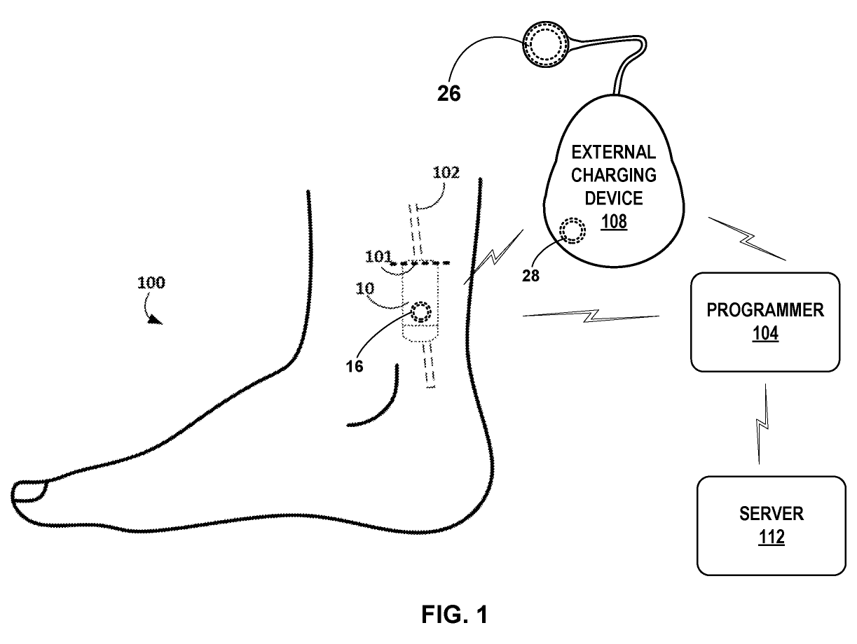

FIG. 1 is a conceptual diagram illustrating an example of a system 100 that includes an IMD 10 and an external device (e.g., a programmer 104 and/or an external charging device 108) that is configured to communicate with IMD 10. In the example of FIG. 1, system 100 includes IMD 10, programmer 104, external charging device 108, and a server 112. In other examples, at least one of the devices may be removed from system 100. For example, system 100 may include IMD 10 and programmer 104, or IMD 10 and external charging device 108, or IMD 10 and any two of programmer 104, external charging device 108, and server 112. Other modifications, such as adding, removing, or duplicating devices, are also within the scope of this disclosure.

External charging device 108 includes one or more charging coils, such as external primary coil 26 or internal primary coil 28. External charging device 108 may be used to program or adjust settings of IMD 10 and may also recharge an electrical energy storage device, such as a battery, of IMD 10. External charging device 108 may also communicate with server 112. In other examples, an external device (e.g., programmer 104) separate from external charging device 108 may communicate with IMD 10 to adjust therapy and/or sensing parameters, download recorded data, or perform other functions.

Server 112 may be one or more servers in a local network or in a cloud computing environment. Server 112 may be configured to communicate with programmer 104, external charging device 108 and/or IMD 10 via wireless communication through a network access point (not shown in FIG. 1) and may be co-located with external charging device 108 and/or programmer 104, or may be located elsewhere, such as in a cloud computing data center.

The example of FIG. 1 is a side view of a patient’s leg showing a leadless neurostimulation IMD 10 near the ankle adjacent to a tibial nerve 102. IMD 10 can be implanted through the patient’s skin and cutaneous fat layer via a small incision 101 (e.g., about one to three centimeters (cm)) above the tibial nerve on a medial aspect of the patient’s ankle. While incision 101 is shown approximately horizontal to the length of the tibial nerve, other incisions or implantation techniques could be used according to physician preference. The example of FIG. 1 describes a neurostimulation implantable medical device for tibial nerve stimulation. In other examples, the techniques of this disclosure may apply to other devices, such as implantable neurostimulation system for use in spinal cord stimulation therapy and deep brain stimulation, as well as to other types of medical devices without limitation.

IMD 10 may be positioned adjacent to the region defined by flexor digitorum longus and soleus in which tibial nerve 102 is contained and implanted adjacent and proximal to a fascia layer. One or more electrodes of IMD 10 may face toward tibial nerve 102. Though not shown in FIG. 1, IMD 10 may also connect to one or more leads comprising one or more electrodes (not shown in FIG. 1).

IMD 10 may be constructed of any polymer, metal, or composite material sufficient to house the components of IMD 10. In some examples, IMD 10 is constructed with a biocompatible housing, such as titanium or stainless steel, or a polymeric material such as silicone or polyurethane, and surgically implanted at a site in patient near the tibial nerve. In other examples, IMD 10 is implanted near the pelvis, abdomen, or buttocks. The housing of IMD 10 may be configured to provide a hermetic seal for components, such as a rechargeable power source. In addition, the housing of IMD 10 may be selected of a material that facilitates receiving energy to charge the rechargeable power source.

Optional testing of neurostimulation IMD 10 may be performed to determine if IMD 10 has been properly positioned in proximity to tibial nerve 102 to elicit a desired response from an applied electrical stimulation. In an example, IMD 10 is controlled by programmer 104 or external charging device 108 to deliver test stimulation, and one or more indicative responses are monitored, such as toe flexion from simulation of the tibial motor neurons controlling the flexor hallucis brevis or flexor digitorum brevis, or a tingling sensation in the heel or sole of the foot excluding the medial arch. If such testing does not elicit appropriate motor or sensory responses, a clinician or other user may reposition IMD 10 and retest.

Once the clinician or other user has determined IMD 10 is properly positioned to provide an appropriate patient response to delivered stimulation therapy, the housing of device can be secured in place as needed. Securing IMD 10 may be optional as the natural shape of the region in which IMD 10 is implanted, and the shape of IMD 10 itself may have good compatibility with the surrounding tissue thus preventing IMD 10 from shifting or rolling after implantation. In some examples, leadless neurostimulation IMD 10 may further include one or more suture points to help secure IMD 10 to fascia or other parts of the patient. In some examples, a suture anchor may be included, such as at the distal end of the housing of IMD 10.

During operation, an electrical stimulation signal may be transmitted between one or more electrodes through the fascia layer. The electrical signal may be used to stimulate tibial nerve 102 which may be useful in the treatment of overactive bladder (OAB) symptoms of urinary urgency, urinary frequency and/or urge incontinence, fecal incontinence, pain, or other symptoms.

In some examples, disease, age, and injury may impair physiological functions of a patient. In one example, bladder dysfunction, such as overactive bladder, urgency, or urinary incontinence, is a problem that may afflict people of all ages, genders, and races. Various muscles, nerves, organs, and conduits within the pelvic floor cooperate to collect, store and release urine. A variety of disorders may compromise urinary tract performance, and contribute to an overactive bladder, urgency, or urinary incontinence that interferes with normal physiological function. System 100 may help relieve some symptoms of some disorders.

Urinary incontinence may include urge incontinence and stress incontinence. In some examples, urge incontinence may be caused by disorders of peripheral or central nervous systems that control bladder micturition reflexes. Some patients may also suffer from nerve disorders that prevent proper triggering and operation of the bladder, sphincter muscles or nerve disorders that lead to overactive bladder activities or urge incontinence. In some cases, urinary incontinence may be attributed to improper sphincter function, either in the internal urinary sphincter or external urinary sphincter.

One type of therapy for treating bladder dysfunction includes delivery of electrical stimulation to a target tissue site within a patient to cause a therapeutic effect during delivery of the electrical stimulation. For example, delivery of electrical stimulation from IMD 10 to a target therapy site, e.g., a tissue site that delivers stimulation to modulate activity of a tibial nerve, spinal nerve (e.g., a sacral nerve), a pudendal nerve, dorsal genital nerve, an inferior rectal nerve, a perineal nerve, or branches of any of the aforementioned nerves, may provide a therapeutic effect for bladder dysfunction, such as a desired reduction in frequency of bladder contractions. In some cases, electrical stimulation of the tibial nerve may modulate afferent nerve activities to restore urinary function.

Bladder dysfunction generally refers to a condition of improper functioning of the bladder or urinary tract, and may include, for example, an overactive bladder, urgency, or urinary incontinence. Overactive bladder (OAB) is a patient condition that may include symptoms, such as urgency, with or without urinary incontinence. Urgency is a sudden, compelling urge to urinate, and may often, though not always, be associated with urinary incontinence. Urinary incontinence refers to a condition of involuntary loss of urine, and may include urge incontinence, stress incontinence, or both stress and urge incontinence, which may be referred to as mixed urinary incontinence. As used in this disclosure, the term “urinary incontinence” includes disorders in which urination occurs when not desired, such as stress or urge incontinence. Other bladder dysfunctions may include disorders such as non-obstructive urinary retention.

In some examples, the techniques described in this disclosure are directed to delivery of neurostimulation therapy in a non-continuous manner which may include on-cycles and off-cycles. For example, an IMD may deliver neurostimulation therapy for a specified period of time followed by a specified period of time when the IMD does not deliver neurostimulation (e.g., withholds delivery of neurostimulation). A period during which stimulation is delivered (an on-cycle) may include on and off periods (e.g., a duty cycle or bursts of pulses) with short inter-pulse durations of time when pulses are not delivered. In some examples, IMD 10 may switch between different operational modes that have different power consumptions for delivery of stimulation and non-delivery of stimulation in order to conserve power when stimulation is not to be delivered. In some examples, a continuous off period may be comparatively long, such as on the order of several days or even several weeks at a time.

The rechargeable power source of IMD 10 may include one or more capacitors, batteries, or other components (e.g., chemical or electrical energy storage devices). Example batteries may include lithium-based batteries, nickel metal-hydride batteries, or other materials. The rechargeable power source may be replenished, refilled, or otherwise capable of increasing the amount of energy stored after energy has been depleted. IMD 10 may include a secondary coil 16, wherein the energy received from secondary coil 16 may be conditioned and/or transformed by a charging circuit. The charging circuit may then send an electrical signal used to charge the rechargeable power source when the power source is fully depleted or only partially depleted.

External charging device 108 may be used to recharge the rechargeable power source within IMD 10 implanted in the patient. External charging device 108 may be a hand-held device, a portable device, or a stationary charging system. External charging device 108 may include components necessary to charge IMD 10 through tissue of the patient. External charging device 108 may include an internal primary coil 28 and external primary coil 26. In other examples, external charging device may only include internal primary coil 28 and omit the use of external primary coil 26, or only include external primary coil 26 and omit the use of internal primary coil 28. External charging device 108 may include a housing to enclose operational components such as a processor, memory, user interface, telemetry module, power source, and charging circuit configured to transmit energy to secondary coil 16 via external primary coil 26 and/or internal primary coil 28. Although a user may control the recharging process with a user interface of external charging device 108, external charging device 108 may alternatively be controlled by another device, e.g., programmer 104, a computing device of server 112 such as a tablet computer, laptop, or other similar computing device. The second external charging device of server 112 may include a computing device with a touch-screen user interface. In other examples, external charging device 108 may be integrated with an external programmer, such as patient programmer 104, which may be carried by the patient.

External charging device 108 and IMD 10 may utilize any wireless power transfer techniques that are capable of recharging the power source of IMD 10 when IMD 10 is implanted within the patient. In some examples, system 100 may utilize inductive coupling between internal primary coil 28 and/or external primary coil 26 of external charging device 108 and one or more secondary coils (e.g., secondary coil 16) of IMD 10. In inductive coupling, internal primary coil 28 is placed near implanted IMD 10 such that internal primary coil 28 is aligned with secondary coil 16 of IMD 10. External charging device 108 may then generate an electrical current in internal primary coil 28 based on a selected power level for charging the rechargeable power source of IMD 10. When either internal primary coil 28 or external primary coil 26 are aligned with secondary coil 16, the electrical current in either internal primary coil 28 or external primary coil 26 may magnetically induce an electrical current in secondary coil 16 within IMD 10. Since secondary coil 16 is associated with and electrically coupled to the rechargeable power source, the induced electrical current may be used to increase the voltage, or charge level, of the rechargeable power source. Although inductive coupling is generally described herein, any type of wireless energy transfer may be used to transfer energy between external charging device 108 and IMD 10.

External primary coil 26 and/or internal primary coil 28 may include a wound wire (e.g., a coil) (not shown in FIG. 1). The coil may be constructed of a wire wound in an in-plane spiral (e.g., a disk-shaped coil). In some examples, this single or even multi-layers spiral of wire may be considered a flexible coil capable of deforming to conform with a non-planar skin surface. The coil may include wires that electrically couple the flexible coil to a power source and a charging module configured to generate an electrical current within the coil. Internal primary coil 28 may be external of the housing of external charging device 108 such that internal primary coil 28 can be placed on the skin of the patient proximal to IMD 10. In some examples, internal primary coil 28 may be disposed on the outside of the housing or even within housing.

Either external primary coil 26 and/or internal primary coil 28 of system 100 may include a heat sink device (not shown in FIG. 1). In the example of system 100, external charging device 108 is the power transmitting unit and IMD 10 is the power receiving unit. IMD 10 may be in a flipped or non-flipped position.

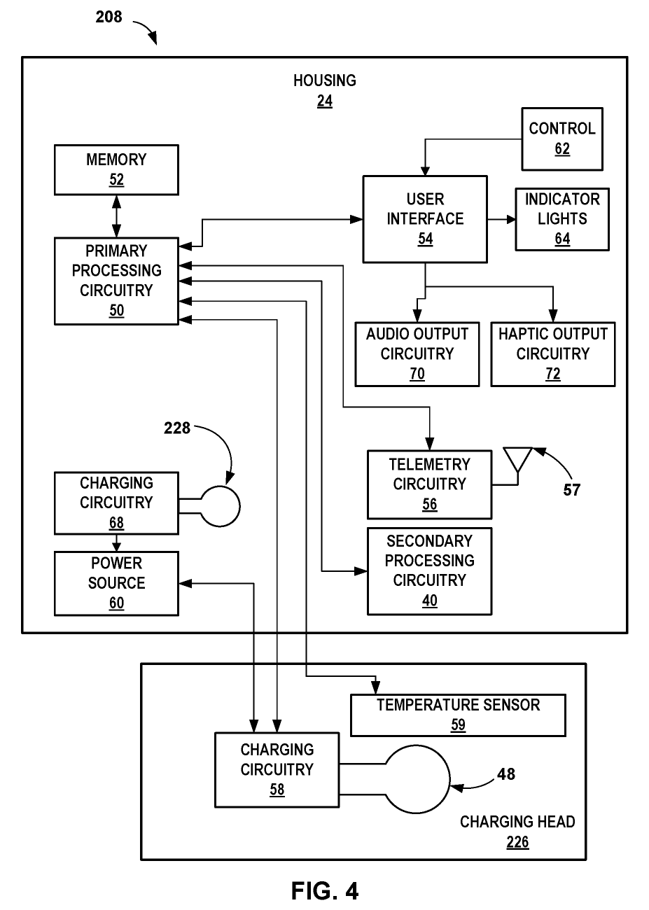

As discussed further in connection with FIG. 4, external charging device 108 may include a user interface to receive control inputs from a user, such as the patient, medical professional, or other caregiver. The user interface of external charging device 108 may also provide information to a user. For example, external charging device 108 may include a control configured to receive user input (not shown in FIG. 1) as well as a set of indicator lights. In some examples the indicator lights may be configured to illuminate the control. The indicator lights may also be configured to output information regarding an operational state of external charging device 108, such as a communication status and wireless power transfer status.

As discussed further in connection with FIG. 4, external charging device 108 includes processing circuitry configured to perform one or more processes related to external charging device 108. In some examples, the processing circuitry determines whether IMD 10 and external charging device 108 have established a communication link e.g., via communication circuitry. In response to the processing circuitry determining that external charging device 108 and IMD 10 have not established a communication link, the processing circuitry may cause a notification to be generated. External charging device 108 may still wirelessly transfer power to IMD 10, but the notification may signify that external charging device 108 is operating in open loop charging mode.

Processing circuitry of external charging device 108 may further determine whether IMD 10 is receiving wireless power. In response to determining that IMD 10 has good power coupling, such as receiving an amount of wireless power above a power threshold, the processing circuitry may cause a notification to be generated.

Processing circuitry of system 100, e.g., processing circuitry of IMD 10, processing circuitry of programmer 104, processing circuitry of external charging device 108, and/or processing circuitry of server 112 may determine (e.g., calculate, receive, lookup, etc.) any of the values described herein.

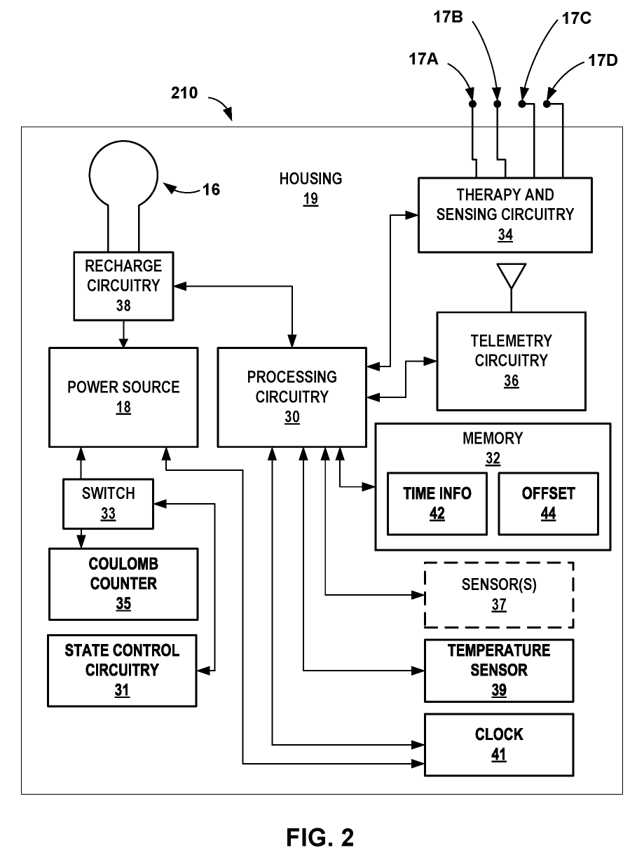

FIG. 2 is a block diagram illustrating example components of the medical device of FIG. 1. Implantable medical device (IMD) 210 is an example of IMD 10 described above in relation to FIG. 1. In the example illustrated in FIG. 2, IMD housing 19 of IMD 210 encloses temperature sensor 39, secondary coil 16, processing circuitry 30, therapy generation and sensing circuitry 34, recharge circuitry 38, memory 32, telemetry circuitry 36, power source 18, switch 33, coulomb counter 35, state control circuitry 31, clock 41, and, in some examples, one or more sensors 37, such as an accelerometer. In other examples, IMD 210 may include a greater or a fewer number of components, e.g., in some examples, IMD 210 may not include temperature sensor 39 or sensors 37. In general, IMD 210 may comprise any suitable arrangement of hardware, alone or in combination with software and/or firmware, to perform the various techniques described herein attributed to IMD 210 and processing circuitry 30, and any equivalents thereof.

Processing circuitry 30 of IMD 210 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 210 may include a memory 32, such as random-access memory (RAM), read only memory (ROM), programmable read only memory (PROM), erasable programmable read only memory (EPROM), electronically erasable programmable read only memory (EEPROM), flash memory, comprising executable instructions for causing the processing circuitry 30 to perform the actions attributed to this circuitry. Moreover, although processing circuitry 30, therapy generation and sensing circuitry 34, recharge circuitry 38, telemetry circuitry 36, temperature sensor 39, state control circuitry 31, coulomb counter 35, switch 33, and clock 41 are described as separate modules, in some examples, some combination of processing circuitry 30, therapy generation and sensing circuitry 34, recharge circuitry 38, telemetry circuitry 36, temperature sensor 39, state control circuitry 31, coulomb counter 35, switch 33, and clock 41 are functionally integrated. In some examples, processing circuitry 30, therapy generation and sensing circuitry 34, recharge circuitry 38, telemetry circuitry 36, and temperature sensor 39, state control circuitry 31, coulomb counter 35, switch 33, and clock 41 correspond to individual hardware units, such as ASICs, DSPs, FPGAs, or other hardware units. In this disclosure, therapy generation and sensing circuitry 34 may be referred to as therapy generation circuitry 34, for simplicity.

Memory 32 may store therapy programs or other instructions that specify therapy parameter values for the therapy provided by therapy generation circuitry 34 and IMD 210. In some examples, memory 32 stores one or more therapy schedules and/or data relating to the transition between therapy schedules.

In some examples, memory 32 may also store time information 42 based on time units (e.g., hours, minutes, seconds, or fractions thereof) counted by the clock 41 from a reference time. In some examples, clock 41 is based on a signal provided by an electronic oscillator, such as a crystal oscillator. The reference time may be a specified or arbitrary point in time that is stored in memory (e.g., at time of factory programming or subsequent reprogramming). The reference time may be “zero” time that corresponds to a particular calendar date and time, in some examples. In some examples, memory 32 may also store an offset 44 to the stored time information 42. Offset 44 may be determined at initialization (e.g., programming by a clinician programmer) to account for differences between the time information of the IMD and local time. Offset 44 may be subsequently updated according to the techniques described herein to account for a daylight savings transition and/or change of time zone (e.g., due to travel), or other such situations that can affect calendar dates and times.

Memory 32 may be configured to store instructions for communication with and/or controlling clock 41. In various examples, memory 32 stores information related to determining the time units counted by clock 41 from the reference time. The time counted by clock 41 from the reference time may be used to determine and store time information 42. As described above, offset 44 is a time offset that may be determined and/or updated by an external device (e.g., programmer 104 and/or external charging device 108). In some examples, offset 44 can be added to or subtracted from time information 42 to calculate a current time according to IMD 210. Offset 44 may be an integer or non-integer number; however, updates to offset 44 will typically be in the form of integer increments/decrements to account for a daylight savings transition and/or a change in time zone.

In some examples, memory 32 may also store temperature data from temperature sensor 39, instructions for recharging rechargeable power source 18, thresholds, instructions for communication between IMD 210 and an external charging device, or any other instructions required to perform tasks attributed to IMD 210. Memory 32 may be configured to store instructions for communication with and/or controlling one or more temperature sensors of temperature sensor 39. In various examples, memory 32 stores information related to determining the temperature of housing 19 and/or exterior surface(s) of housing 19 of IMD 210 based on temperatures sensed by one or more temperature sensors, such as temperature sensor 39, located within IMD 210.

In some examples, memory 32 stores programming settings such as electrical stimulation therapy output magnitude, pulse width, as well as other therapy parameters for one or more therapy programs and/or therapy schedules. Memory 32 may determine whether a sensed bioelectrical signal is valid, such as an evoked compound action potential (ECAP) or other signal in response to an output electrical stimulation therapy event. Memory 32 may store programming instructions that when executed by processing circuitry 30 cause processing circuitry 30 to cause therapy generation circuitry 34 to deliver electrical stimulation therapy to a target nerve of a patient.

In some examples, memory 32 stores data related to power source 18. In some examples, memory 32 stores data of one or more instances of therapy delivery, a status of power source 18 (e.g., an estimated level of charge remaining or measured level of charge remaining), predicted future use, and/or drain of power source 18.

Therapy generation and sensing circuitry 34 may generate and deliver electrical stimulation under the control of processing circuitry 30. In some examples, processing circuitry 30 controls therapy generation circuitry 34 by accessing memory 32 to selectively access and load at least one of the stimulation programs to therapy generation circuitry 34. For example, in operation, processing circuitry 30 may access memory 32 to load one of the stimulation programs to therapy generation circuitry 34. In such examples, relevant stimulation parameters may include a voltage amplitude, a current amplitude, a pulse rate, a pulse width, a duty cycle, or the combination of electrodes 17A, 17B, 17C, and 17D (collectively “electrodes 17”) that therapy generation circuitry 34 may use to deliver the electrical stimulation signal as well as sense biological signals. In other examples, IMD 210 may have more or fewer electrodes than the four shown in the example of FIG. 2. In some examples, electrodes 17 may be part of or attached to a housing of IMD 210, e.g., a leadless electrode. In other examples, one or more of electrodes 17 may be part of a lead implanted in or attached to a patient to sense biological signals and/or deliver electrical stimulation, as described above in relation to FIG. 1.

In some examples, one or more electrodes 17 connected to therapy generation circuitry 34 may connect to one or more sensing electrodes, e.g., attached to housing of IMD 210. In some examples, electrodes 17 may be configured to detect the evoked motor response caused by the electrical stimulation therapy event, or other bioelectrical signals such as ECAPs, impedance, or other signals as appropriate.

IMD 210 also includes components to receive power to recharge rechargeable power source 18 when rechargeable power source 18 has been at least partially depleted. As shown in FIG. 2, IMD 210 includes secondary coil 16 and recharge circuitry 38 coupled to rechargeable power source 18. Recharge circuitry 38 may be configured to charge rechargeable power source 18 with the selected power level determined by either processing circuitry 30 or an external charging device, such as external charging device 108 described above in relation to FIG. 1. Recharge circuitry 38 may include any of a variety of charging and/or control circuitry configured to process or convert current induced in secondary coil 16 into charging current to charge power source 18.

Secondary coil 16 may include a coil of wire or other device capable of inductive coupling with a primary coil disposed external to a patient. Although secondary coil 16 is illustrated as a simple loop of in FIG. 2, secondary coil 16 may include multiple turns of conductive wire. Secondary coil 16 may include a winding of wire configured such that an electrical current can be induced within secondary coil 16 from a magnetic field. The induced electrical current may then be used to recharge rechargeable power source 18.

Recharge circuitry 38 may include one or more circuits that process, filter, convert and/or transform the electrical signal induced in the secondary coil to an electrical signal capable of recharging rechargeable power source 18. For example, in alternating current induction, recharge circuitry 38 may include a half-wave rectifier circuit and/or a full-wave rectifier circuit configured to convert alternating current from the induction to a direct current for rechargeable power source 18. The full-wave rectifier circuit may be more efficient at converting the induced energy for rechargeable power source 18. However, a half-wave rectifier circuit may be used to store energy in rechargeable power source 18 at a slower rate. In some examples, recharge circuitry 38 may include both a full-wave rectifier circuit and a half-wave rectifier circuit such that recharge circuitry 38 may switch between each circuit to control the charging rate of rechargeable power source 18 and temperature of IMD 210.

Rechargeable power source 18 may include one or more capacitors, batteries, and/or other energy storage devices. Rechargeable power source 18 may deliver operating power to the components of IMD 210. In some examples, rechargeable power source 18 may include a power generation circuit to produce the operating power. Rechargeable power source 18 may be configured to operate through many discharge and recharge cycles. Rechargeable power source 18 may also be configured to provide operational power to IMD 210 during the recharge process. In some examples, rechargeable power source 18 may be constructed with materials to reduce the amount of heat generated during charging. In other examples, IMD 210 may be constructed of materials and/or using structures that may help dissipate generated heat at rechargeable power source 18, recharge circuitry 38, and/or secondary coil 16 over a larger surface area of the housing of IMD 210. In some examples, power source 18 includes a non-rechargeable power source.

Although rechargeable power source 18, recharge circuitry 38, and secondary coil 16 are shown as contained within the housing of IMD 210, in other examples, at least one of these components may be disposed outside of the housing. For example, in some implementations, secondary coil 16 may be disposed outside of the housing of IMD 210 to facilitate better coupling between secondary coil 16 and the primary coil of external charging device. In other examples, power source 18 may be a primary power cell and IMD 210 may not include recharge circuitry 38 and secondary coil 16.

Processing circuitry 30 may also control the exchange of information with an external device using telemetry circuitry 36. Telemetry circuitry 36 may be configured for wireless communication using radio frequency (RF) protocols, such as Bluetooth, including Bluetooth low energy (BLE), or similar RF protocols, as well as using inductive communication protocols. Telemetry circuitry 36 may include one or more antennas configured to communicate with an external device (e.g., programmer 104 and/or external charging device 108 of FIG. 1). Processing circuitry 30 may transmit operational information and receive therapy programs or therapy parameter adjustments via telemetry circuitry 36. Also, in some examples, IMD 210 may communicate with other implanted devices, such as stimulators, control devices, or sensors, via telemetry circuitry 36. In addition, telemetry circuitry 36 may be configured to control the exchange of time information 42 and/or offset 44 data. Telemetry circuitry 36 may also be configured to control the exchange of information related to sensed and/or determined temperature data, for example temperatures sensed by and/or determined from temperatures sensed using temperature sensor 39. In some examples, telemetry circuitry 36 may communicate using inductive communication, and in other examples, telemetry circuitry 36 may communicate using RF frequencies separate from the frequencies used for inductive charging.

In some examples, processing circuitry 30 is configured to perform one or more of the timekeeping functions described herein. For example, processing circuitry 30 may be configured to determine time information 42 for IMD 210 based on an internal clock (e.g., clock 41). In some examples, processing circuitry 30 is configured to determine time information 42 based on time units (e.g., hours, minutes, seconds, or fractions thereof) counted by the clock 41 from the reference time. As noted previously herein, the reference time may be a specified or arbitrary point in time that is stored in memory 32 (e.g., at time of factory programming or subsequent reprogramming). At initialization (e.g., programming by a clinician programmer), processing circuitry 30 may be configured to control telemetry circuitry 36 to transmit time information 42 to an external device (e.g., programmer 104) that is configured to determine an offset (i.e., offset 44) to time information 42 to account for differences between time information 42 of IMD 210 and local time. Processing circuitry 30 may be configured to receive, from the external device, offset 44 and may be further configured to store offset 44 in memory 32. In some examples, processing circuitry 30 is configured to determine the current time according to IMD 210 by adding offset 44 to, or subtracting offset 44 from, time information 42. Processing circuitry 30 may be configured to use the current time according to IMD 210, based on time information 42 and offset 44, to control delivery of therapy by IMD 210 according to one or more therapy programs and/or therapy schedules stored in memory 32.

In some examples, processing circuitry 30 is configured to update offset 44 based on internal calculations. For example, processing circuitry 30 is configured to determine a date based on time information 42 and offset 44. Processing circuitry 30 may be configured to compare the date with a daylight savings transition date stored in memory 32. Responsive to determining that the date exceeds the daylight savings transition date, processing circuitry 30 is configured to adjust (e.g., increase/decrease) offset 44 by one (or more) hours to result in correct time information.

In some examples, processing circuitry 30 is configured to update offset 44 based on an updated offset value or offset adjustment value received from an external device (e.g., programmer 104). Instead of relying on actual time updates, wherein timekeeping circuitry is periodically recalibrated to match an external source (e.g., time information from server), IMD 210 saves power and avoids unreliable sources by maintaining internal timekeeping configurations and making limited adjustments to offset 44 (e.g., increasing/decreasing the offset by an integer number of time zone offset intervals, such as hours or fractions thereof) to reflect a change in local time due to travel (i.e., change of time zone) and/or transition from standard time to daylight savings time (DST) or vice versa.

Processing circuitry 30 may be configured to receive, from an external device (e.g., programmer 104), a request for time and offset information. Responsive to receiving the request for the time and offset information, processing circuitry 30 may be configured control telemetry circuitry 36 to transmit time information 42 and offset 44 to the external device. For example, processing circuitry 30 may be configured to send time information 42 and offset 44 as separate values or as a combined value (e.g., transmitting, to the external device, the current time according to IMD 210). In other examples, processing circuitry 30 may send time information 42 without offset 44, and the received offset value from the external device can still be used to replace or adjust offset 44 according to the received offset value from the external device.

In some examples, processing circuitry 30 is configured to receive, from an external device (e.g., programmer 104), daylight savings transition information. Responsive to receiving the daylight savings transition information, processing circuitry 30 is configured to adjust offset 44 by one hour. For example, the external device may transmit daylight savings transition information (e.g., a one-hour offset adjustment or an updated offset value that reflects a one-hour adjustment) after determining, via processing circuitry of the external device, that a daylight savings transition has occurred based on time information 42 and offset 44 received from IMD 210. In other examples, the external device may transmit daylight savings transition information based on a user input indication or otherwise received or determined indication that a daylight savings transition has occurred. In some examples, processing circuitry 30 is configured to apply to offset 44 a one-hour offset adjustment received from the external device, via telemetry circuitry 36. In other examples, processing circuitry 30 is configured to replace the stored value of offset 44 with an updated offset value received from the external device, via telemetry circuitry 36, that reflects a one-hour adjustment.

In some examples, processing circuitry 30 is configured to receive, from an external device (e.g., programmer 104), updated time zone information. Responsive to receiving the updated time zone information, processing circuitry 30 is configured to adjust offset 44 by an integer number of time zone offset intervals, such as hours or fractions thereof. For example, the external device may transmit updated time zone information (e.g., an N-hour offset adjustment or an updated offset value that reflects an N-hour adjustment, where N is an integer value) after determining, via processing circuitry of the external device, that a difference of N hours exists between a user input time zone and a previous time zone of IMD 210 based on a comparison between the user input time zone and time zone information (e.g., predetermined time zone, or time zone derived from time information 42 and offset 44) received from IMD 210. In other examples, the external device may transmit updated time zone information based on a user input location or otherwise received or detected location information from which an applicable time zone can be derived. In some examples, processing circuitry 30 is configured to apply to offset 44 an N-hour offset adjustment received from the external device, via telemetry circuitry 36. In other examples, processing circuitry 30 is configured to replace the stored value of offset 44 with an updated offset value received from the external device, via telemetry circuitry 36, that reflects an N-hour adjustment.

In some examples, daylight savings time adjustments or time zone adjustments, or both, can be turned off based on user preference or for jurisdictions that do not observe daylight savings time. For example, processing circuitry 30 may be configured to receive, via a user interface, a user input requesting toggling on or off daylight savings time adjustments or time zone adjustments, or both. Responsive to the user input, processing circuitry 30 may be configured to toggle on/off the daylight savings time adjustments or the time zone adjustments, or both. For example, a user input to toggle on/off daylight savings time or time zone adjustments may be entered via a user interface of an external device (e.g., programmer 104). Responsive to the user input, the external device may be configured to transmit an instruction to IMD 210 that causes processing circuitry 30 to toggle on/off daylight savings time or time zone adjustments. In other examples, toggling on/off daylight savings time adjustments or time zone adjustments is performed entirely by processing circuitry of the external device.

In some examples, processing circuitry 30 is configured to retrieve, from memory 32, information of one or more therapy programs for IMD 210 (e.g., therapy parameters and a therapy schedule defining delivery of therapy by IMD 210). Processing circuitry 30 is configured to control IMD 210 to deliver therapy according to time information 42, offset 44, and a therapy program that at least partially defines the therapy. Because the system relies on limited updates to offset 44 and leaves time information 42 unchanged, IMD 210 can deliver therapy consistently according to a programmed therapy schedule without ever needing information about true time. The system may run entirely on internal timekeeping capabilities with offset adjustments to account for daylight savings transitions and time zone changes, but no need for calibration with any external time source after initialization.

In some examples, processing circuitry 30 may be configured to safeguard against therapy occurring at a higher frequency than intended (e.g., due to large time zone changes). For example, processing circuitry 30 may also be configured to determine a time interval between a scheduled therapy session and a previously delivered therapy session. Processing circuitry 30 may be configured to compare the time interval with a waiting period (e.g., 24 hours or any other specified waiting period). Responsive to determining that the waiting period exceeds the time interval, processing circuitry 30 may control IMD 210 to withhold the scheduled therapy session.

In some examples, processing circuitry 30 may transmit, via control of telemetry circuitry 36, information to external charging device related to the operation of rechargeable power source 18. For example, processing circuitry 30 may control telemetry circuitry 36 to transmit indications that rechargeable power source 18 is completely charged, rechargeable power source 18 is fully discharged, the amount of charging current output by recharge circuitry 38 e.g., to power source 18, or any other charge status of rechargeable power source 18. In some examples, processing circuitry 30 may use telemetry circuitry 36 to transmit instructions to external charging device, including instructions regarding further control of the charging session, for example instructions to lower the power level or to terminate the charging session, based on the determined temperature of IMD housing 19.

Processing circuitry 30 may also transmit information to external charging device that indicates any problems or errors with rechargeable power source 18 that may prevent rechargeable power source 18 from providing operational power to the components of IMD 210. In various examples, processing circuitry 30 may receive, through telemetry circuitry 36, instructions for algorithms, including formulas and/or values for constants to be used in the formulas, that may be used to determine the temperature of the housing 19 and/or exterior surface(s) of housing 19 of IMD 210 based on temperatures sensed by temperature sensor 39 located within IMD 210 during and after a recharging session performed on rechargeable power source 18.

IMD 210 also includes components for determining a status of power source 18. For example, in examples where power source 18 includes a battery, IMD 210 may include components for determining (e.g., measuring, estimating, receiving, etc.) information related to a battery status, battery level, and/or other battery information (e.g., an amount of current drain from the battery, an amount of charge remaining in the battery, etc.). Components of IMD 210 for determining information related to the battery status include coulomb counter 35, switch 33, clock 41, and state control circuitry 31. Coulomb counter 35, switch 33, clock 41, and state control circuitry 31 may be used alone and/or in connection with other components of IMD 210, including processing circuitry 30.

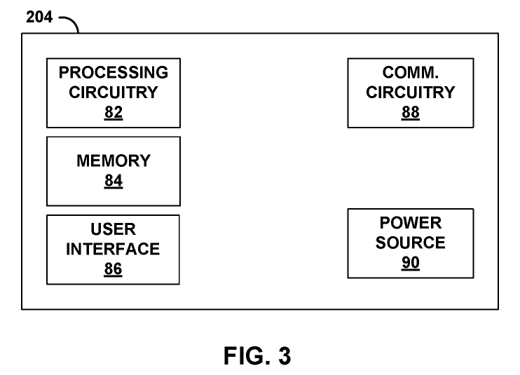

FIG. 3 is a block diagram of an example programmer of FIG. 1. Programmer 204 is an example of programmer 104 described above in relation to FIG. 1. Programmer 204 may be a device for inputting information relating to a patient, receiving information from IMD 210, and updating IMD 210. In some examples, such as where programmer 204 is a patient programmer, programmer 204 can be a wearable communication device, with a therapy request input integrated into a key fob or a wristwatch, handheld computing device, smart phone, computer workstation, or networked computing device. Programmer 204 can be a bring-your-own device provided by the patient, or provided by the healthcare provider in connection with the implantable device.

In some examples, such as where programmer 204 is physician/clinician programmer, programmer 204 is a tablet computing device that is preloaded with a specific application to interface with IMD 210. The physician or clinician may interact with programmer 204 for programming IMD 210. The physician or clinician may utilize programmer 204 to program IMD 210 (e.g., program therapy parameters, such as for one or more therapy schedules), as well as view information about the usage of IMD 210.

Programmer 204 generally comprises a processing circuitry 82, a memory 84, a user interface 86, communications circuitry 88, and a power source 90. Processing circuitry 82 can be any programmable device that accepts digital data as input, is configured to process the input according to instructions or algorithms, and provides results as outputs. In an example, processing circuitry 82 can be a central processing unit (CPU) configured to carry out the instructions of a computer program. Processing circuitry 82 is therefore configured to perform at least basic arithmetical, logical, and input/output operations. In one or more examples, processing circuitry 82 corresponds to individual hardware units, such as microprocessors, ASICs, DSPs, FPGAs, or other hardware units. In other examples, processing circuitry 82 can correspond to multiple individual hardware units, such as microprocessors, ASICs, DSPs, FPGAs, or other hardware units.

Memory 84 can comprise volatile or non-volatile memory as required by processing circuitry 82 to not only provide space to execute the instructions or algorithms, but to provide the space to store the instructions themselves. In one or more examples, volatile memory can include RAM, DRAM, or static random access memory (SRAM), for example. In one or more examples, non-volatile memory can include read-only memory, flash memory, ferroelectric RAM, hard disk, floppy disk, magnetic tape, or optical disc storage, for example. The foregoing lists in no way limit the type of memory that can be used.

User interface 86 can include a button or keypad, lights, a speaker for voice commands, a knob able to turn, a display, such as a liquid crystal display (LCD), light-emitting diode (LED), or cathode ray tube (CRT). In some examples, the display may be a touch screen. Processing circuitry 82 can present and receive information relating to electrical stimulation and resulting therapeutic effects via user interface 86. For example, processing circuitry 82 can receive patient input via user interface 86. The input can be, for example, in the form of pressing a button on a keypad or selecting an icon from a touch screen. Processing circuitry 82 can also present information to the patient in the form of alerts related to delivery of the electrical stimulation to a patient or a caregiver via user interface 86. In some examples, user interface 86 is configured to provide time according to time information 42 and offset 44 stored in memory 32 of IMD 210 and/or receive a user input (e.g., time zone selection or request to toggle on/off DST) to update offset 44.

Communication circuity 88 is configured to interface with IMD 210 and optionally, server 112 (FIG. 1). Communication circuity 88 supports wireless communication between programmer 204 under the control of processing circuitry 82 and IMD 10 (e.g., IMD 210 via telemetry circuitry 36 or another communication interface) and, optionally, external charging device 108 and/or server 112. Communication circuity 88 can also be configured to communicate with another computing device via wireless communication techniques, or direct communication through a wired connection. Communication circuity 88 can provide wireless communication via an RF or proximal inductive medium. In some examples, communication circuity 88 can include an antenna, which may take on a variety of forms, such as an internal or external antenna.

Examples of local wireless communication techniques that may be employed to facilitate communication between programmer 204 and another computing device include RF communication according to the 802.11 or Bluetooth specification sets, infrared communication, e.g., according to the Infrared Data Association (IrDA) standard, or other standard or proprietary telemetry protocols. In this manner, other external devices may be capable of communicating with programmer 204 without needing to establish a secure wireless connection.

Power source 90 delivers operating power to the components of programmer 204. Power source 90 can include a battery and a power generation circuit to produce the operating power. In some examples, the battery may be rechargeable by for example, an exterior power source.

Programmer 204 allows the user (e.g., patient, caretaker, clinician, physician) to program one or more therapy schedules and therapy parameters (e.g., amplitude, frequency, and/or pulse width) according to one or more therapy programs. A therapy schedule may include a frequency and duration of stimulation therapy based on certain time intervals (e.g., times of the day, number of days between therapy sessions, particular dates, and/or days of the week for therapy sessions, total duration, and/or number of therapy sessions, etc.). Programmer 204 can communicate with IMD 210 to update the functionality of IMD 210. In this way, programmer, e.g., via processing circuitry 82, is configured to control IMD 210 to deliver electrical stimulation therapy, including therapy to one or more of a sacral nerve or tibial nerve for incontinence therapy according to the one or more therapy programs. In some examples, programmer 204 is configured to communicate with external charging device 208, which may in turn communication with IMD 210 for programming and/or controlling IMD 210.

In some examples, programmer 204 is configured to perform one or more of the functions related to updating an offset to time information for IMD 210, as described above. For example, programmer 204, via processing circuitry 82, may be configured to determine whether offset 44 needs to be adjusted to account for a daylight savings transition or change of time zone. In some examples, programmer 204 is configured to determine an adjusted value for offset 44 (e.g., increase or decrease offset 44 by an integer number of time zone offset intervals, such as hours or fractions thereof) to account for the daylight savings transition and/or change of time zone. The programmer 204 may be configured to control communication circuitry 88 to send the adjusted value for offset 44 to be stored in memory 32 of IMD 210 (i.e., in place of a previously stored value for offset 44). Instead of determining an adjusted value for offset 44, in some examples, programmer 204 may be configured to determine an adjustment (e.g., +/- integer number of hours or fractions thereof) needed for offset 44 to account for the daylight savings transition and/or change of time zone. The programmer 204 may be configured to control communication circuitry 88 to send the adjustment for offset 44 to IMD 210, wherein the adjustment is implemented (e.g., added to or subtracted from offset 44) by processing circuitry 30 of IMD 210.

Any of the time offset computation or therapy scheduling tasks described in relation to IMD 210 or programmer 204 may be performed by at least partially performed by processing circuitry 82 instead of or in support of processing circuitry 30. For example, functions that require comparatively high energy and/or processor usage be performed by processing circuitry 82 of programmer 204 to conserve power and/or processor bandwidth in IMD 210.

In some examples, processing circuitry 82 generates, for output to a user, one or more prompts related to a status of power source 18 of IMD 210. In some examples, processing circuitry 82 generates, for output to a user, a prompt to recharge. In some examples, where processing circuitry 82 receives an indication that power source 18 has low energy (e.g., energy below a certain predefined threshold), such that power source 18 needs to be recharge immediately, processing circuitry 82 can generate a prompt to charge immediately (e.g., today).

FIG. 4 is a block diagram of an example of an external charging device of FIG. 1. External charging device 208 of FIG. 4 is an example of external charging device 108 described above in relation to FIG. 1. In some examples, external charging device 208 may be described as a hand-held device, in other examples, external charging device 208 may be a larger or a non-portable device. In addition, in other examples external charging device 208 may be included as part of an external programmer or include functionality of an external programmer. As shown in the example of FIG. 4, external charging device 208 includes a housing 24 connected to a charging head 226. Housing 24 encloses components such as a primary processing circuitry 50, memory 52, user interface 54, telemetry circuitry 56, control 62, one or more sets of indicator lights 64, audio output circuitry 70, haptic output circuitry 72 and power source 60. Charging head 226 may include charging circuitry 58, temperature sensor 59, and external primary coil 48. Charging head 226 and/or external primary coil 48 may be an example of external primary coil 26 as shown in FIG. 1. Housing 24 is electrically coupled to charging head 226 via a cable. Housing 24 may also include charging circuitry 68 and internal primary coil 228, which is an example of internal primary coil 28 described above in relation to FIG. 1.

In some examples, separate charging head 226 may facilitate positioning of external primary coil 48 over secondary coil 16 of IMD 10 (as shown in FIG. 1) or IMD 210 (as shown in FIG. 2). In some examples, charging circuitry 68 and/or internal primary coil 228 may be integrated within housing 24. In other examples, external charging device 208 may not include charging head 226. Memory 52 may store instructions that, when executed by primary processing circuitry 50, causes primary processing circuitry 50 and external charging device 208 to provide the functionality ascribed to external charging device 208 throughout this disclosure, and/or any equivalents thereof. External primary coil 48 and internal primary coil 228 may also be referred to as an antenna. In some examples, external charging device 208 may include secondary processing circuitry 40, which may control telemetry circuitry 56, as well as perform other functions. Some other functions may include error checking of the operation of primary processing circuitry 50.

External charging device 208 may also include one or more temperature sensors, illustrated as temperature sensor 59 within charging head 226, similar to temperature sensor 39 of FIG. 2. As shown in FIG. 4, temperature sensor 59 may be disposed within charging head 226. In other examples, one or more temperature sensors of temperature sensor 59 may be disposed within housing 24. For example, charging head 226 may include one or more temperature sensors positioned and configured to sense the temperature of external primary coil 48 and/or a surface of the housing of charging head 226. In some examples, external charging device 208 may not include temperature sensor 59.

In general, external charging device 208 comprises any suitable arrangement of hardware, alone or in combination with software and/or firmware, to perform the techniques ascribed to external charging device 208, and primary processing circuitry 50, user interface 54, telemetry circuitry 56, and charging circuitry 68 of external charging device 208, and/or any equivalents thereof. In various examples, external charging device 208 may include one or more processors, such as one or more microprocessors, DSPs, ASICs, FPGAs, or any other equivalent integrated or discrete logic circuitry, as well as any combinations of such components. External charging device 208 also, in various examples, may include a memory 52, such as RAM, ROM, PROM, EPROM, EEPROM, flash memory, a hard disk, a CD-ROM, comprising executable instructions for causing the one or more processors to perform the actions attributed to them. Moreover, although primary processing circuitry 50, telemetry circuitry 56, charging circuitry 68, and temperature sensor 59 are described as separate modules, in some examples, primary processing circuitry 50, telemetry circuitry 56, charging circuitry 68, and/or temperature sensor 59 are functionally integrated. In some examples, primary processing circuitry 50, telemetry circuitry 56, charging circuitry 68, and/or temperature sensor 59 correspond to individual hardware units, such as ASICs, DSPs, FPGAs, or other hardware units.

Memory 52 may store instructions that, when executed by primary processing circuitry 50, cause primary processing circuitry 50 and external charging device 208 to provide the functionality ascribed to external charging device 208 throughout this disclosure, and/or any equivalents thereof. For example, memory 52 may include instructions that cause primary processing circuitry 50 to control the power level used to charge IMD 210 in response to the determined temperatures for the housing/external surface(s) of IMD 210, as communicated from IMD 210, or instructions for any other functionality. Memory 52 may include a record of selected power levels, sensed temperatures, determined temperatures, or any other data related to charging rechargeable power source 18, described above in relation to FIG. 2. Memory 52 may store instructions that when executed by primary processing circuitry 50 may control the operation of indicator lights 64 as described above in relation to FIG. 1. Primary processing circuitry 50 may determine one or more operational states, e.g., of external charging device 208 and selectively control indicator lights 64 based on the operational state.

Primary processing circuitry 50 may, when requested, transmit any stored data in memory 52 to another computing device for review or further processing, such as to server 112 depicted in FIG. 1. Primary processing circuitry 50 may be configured to access memory, such as memory 32 of IMD 10 and/or memory 52 of external charging device 208, to retrieve information comprising instructions, formulas, and determined values for one or more constants.

User interface 54 may include buttons, such as control 62 or a keypad, lights, such as indicator lights 64, a speaker for voice commands, a display, such as a liquid crystal display (LCD), light-emitting diode (LED), or cathode ray tube (CRT). In some examples, the display may be a touch screen. Control 62 may be implemented as any type of component that may receive user input and provide an indication of the user input to primary processing circuitry 50. Control 62 may be a knob, switch, button, or another suitable structure. As discussed in this disclosure, primary processing circuitry 50 may present and receive information relating to the charging and/or the status of rechargeable power source 18 (e.g., a battery) of IMD 210 via user interface 54. For example, user interface 54 may indicate when charging is occurring, quality of the alignment between internal primary coil 228 or external primary coil 48 and secondary coil 16 of IMD 210, the selected power level, current charge level of rechargeable power source 18, duration of the current recharge session, anticipated remaining time of the charging session, sensed temperatures, or any other information. Primary processing circuitry 50 may receive some of the information displayed on user interface 54 from IMD 210 in some examples. In some examples, user interface 54 may provide an indication to the user of the status of power source 18 of IMD 210. For example, user interface 54 may provide information indicative of the status of power source 18 (e.g., a battery status) including an indication of at least one of an amount (e.g., a percentage) of remaining charge, a time until recharge, an expected date of battery depletion, or an expected date of battery recharge.

User interface 54 may also receive user input via user interface 54. The input may be, for example, in the form of pressing a button on a keypad or selecting an icon from a touch screen. The input may change programmed settings, start or stop therapy, request starting or stopping a recharge session, a desired level of charging, or one or more statistics related to charging rechargeable power source 18 (e.g., the cumulative thermal dose). In this manner, user interface 54 may allow the user to view information related to the operation of IMD 210. For example, control 62 may provide an input to primary processing circuitry 50 to cause primary processing circuitry 50 to start or stop delivery of wireless power to the power receiving device, e.g., IMD 10 or IMD 210 described above in relation to FIGS. 1 and 2.

Charging circuitry 58 may include one or more circuits that generate an electrical signal, and an electrical current, within external primary coil 48. Charging circuitry 58 may generate an alternating current of specified amplitude and frequency in some examples. In other examples, charging circuitry 58 may generate a direct current. In any case, charging circuitry 58 may be capable of generating electrical signals, and subsequent magnetic fields, to transmit various levels of power to IMD 210. In this manner, charging circuitry 58 may be configured to charge rechargeable power source 18 of IMD 210 with the selected power level.

Power source 60 may deliver operating power to the components of external charging device 208. Power source 60 may also deliver the operating power to drive external primary coil 48 during the charging process. Power source 60 may include a battery and a power generation circuit to produce the operating power. In some examples, a battery of power source 60 may be rechargeable to allow extended portable operation. In other examples, power source 60 may draw power from a wired voltage source such as a consumer or commercial power outlet.

Telemetry circuitry 56 supports wireless communication between IMD 210 and external charging device 208 under the control of primary processing circuitry 50. Telemetry circuitry 56 may also be configured to communicate with another computing device via wireless communication techniques, or direct communication through a wired connection. In some examples, telemetry circuitry 56 may be substantially similar to telemetry circuitry 36 of IMD 210 described herein, providing wireless communication via an RF or proximal inductive medium. In some examples, telemetry circuitry 56 includes an antenna 57, which may take on a variety of forms, such as an internal or external antenna. Although telemetry circuitry 56 and telemetry circuitry 36 may each include dedicated antennas for communications between these devices, telemetry circuitry 56 and telemetry circuitry 36 may instead, or additionally, be configured to utilize inductive coupling from internal primary coil 228 and/or external primary coil 48 to transfer data.

Examples of local wireless communication techniques that may be employed to facilitate communication between external charging device 208 and IMD 210 include radio frequency and/or inductive communication according to any of a variety of standard or proprietary telemetry protocols, or according to other telemetry protocols such as the Institute of Electrical and Electronics Engineers (IEEE) 802.11x or Bluetooth specification sets. In this manner, other external devices may be capable of communicating with external charging device 208 without needing to establish a secure wireless connection.

In operation, primary processing circuitry 50, and/or secondary processing circuitry 40, may control one or more sets of indicator lights 64 to provide information to a user about communication, charging efficiency, therapy status of the IMD, or other applicable information. For example, primary processing circuitry 50 may determine whether communication circuitry, e.g., telemetry circuitry 56, has established a communication link with a power receiving device (e.g., IMD 10 or IMD 210 depicted in FIGS. 1 and 2). Primary processing circuitry 50 may also determine whether the power receiving device (e.g., IMD 10 or IMD 210) is receiving wireless power, e.g., via charging circuitry 68 and internal primary coil 228, or charging circuitry 58 and external primary coil 48.

In some examples, one or more functions of programmer 204 are integrated within or alternatively performed by external charging device 208. For example, any of the time offset computation or therapy scheduling tasks described in relation to IMD 210 or programmer 204 may be performed by at least partially performed by processing circuitry 50 instead of or in support of processing circuitry 30 and processing circuitry 82.

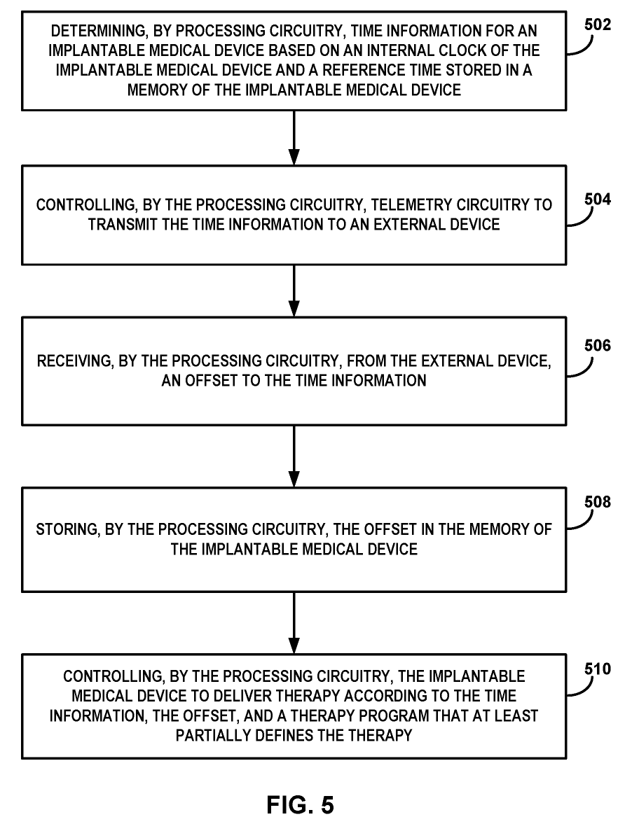

FIG. 5 is a flow chart illustrating an example technique for setting time information and an offset to time information for an IMD. The example technique of FIG. 5 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 5.

In the example of FIG. 5, processing circuitry 30 determines time information 42 for IMD 210 based on an internal clock (e.g., clock 41) and a reference time (502). As discussed herein, processing circuitry 30 may be configured to determine time information 42 based on time units (e.g., hours, minutes, seconds, or fractions thereof) counted by the clock 41 from the reference time, wherein the reference time may be a specified or arbitrary point in time that is stored in memory 32 (e.g., at time of factory programming or subsequent reprogramming).

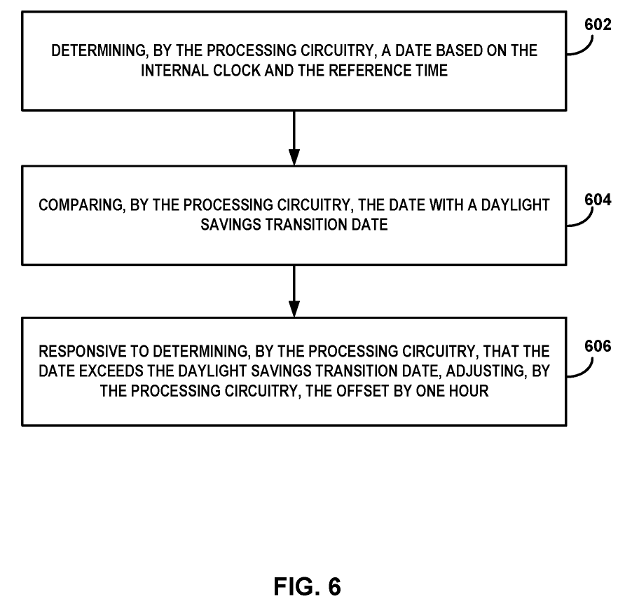

Processing circuitry 30 controls telemetry circuitry 36 to transmit time information 42 to an external device (504). For example, processing circuitry 30 may be configured to control telemetry circuitry 36 to transmit time information 42 to programmer 204 (e.g., where programmer 204 is a clinician programmer). Processing circuitry 82 of programmer 204 may be configured to determine an offset (i.e., offset 44) to time information 42 to account for differences between time information 42 of IMD 210 and local time (i.e., according to time information stored in memory of programmer 204 and/or obtained from a trustworthy source, e.g., a server linked to an atomic clock). In some examples, offset 44 is determined in this manner at initialization (e.g., initial programming or subsequent reprogramming in a clinical setting by programmer 204, where programmer 204 is a clinician programmer). Thereafter, offset 44 may be updated according to techniques described herein with reference to FIGS. 6 through 9.

Processing circuitry 30 receives offset 44 from the external device (e.g., programmer 204) via telemetry circuitry 36 (506). For example, processing circuitry 30 may be configured to control telemetry circuitry 36 to receive a value for offset 44 from communication circuitry 82 of programmer 204 that is under control of processor 82. Processing circuitry 30 then stores offset 44 in memory 32 (508).

Processing circuitry may control IMD 210 to deliver therapy according to time information 42, offset 44, and a therapy program that at least partially defines the therapy (510). For example, processing circuitry 30 may determine time (i.e., the current time according to IMD 210) by adding offset 44 to, or subtracting offset 44 from, time information 42, and may be configured to execute one or more therapy programs and/or therapy schedules stored in memory 32 based on time determined in this manner.

FIG. 6 is a flow chart illustrating an example technique for updating an offset to time information for an IMD. The example technique of FIG. 6 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 6.

In some examples, after time information 42 and offset 44 are set using a technique such as the technique described in relation to FIG. 5, offset 44 may be updated to adjust for daylight savings transitions based on internal calculations of IMD 210 (e.g., as described in relation to FIG. 6).

In the example of FIG. 6, processing circuitry 30 determines a date based on time information 42 and offset 44 (602). Processing circuitry 30 compares the date with a daylight savings transition date stored in memory 32 (604). Responsive to determining that the date exceeds the daylight savings transition date, processing circuitry 30 adjusts (e.g., increase/decrease) offset 44 by one hour (606).

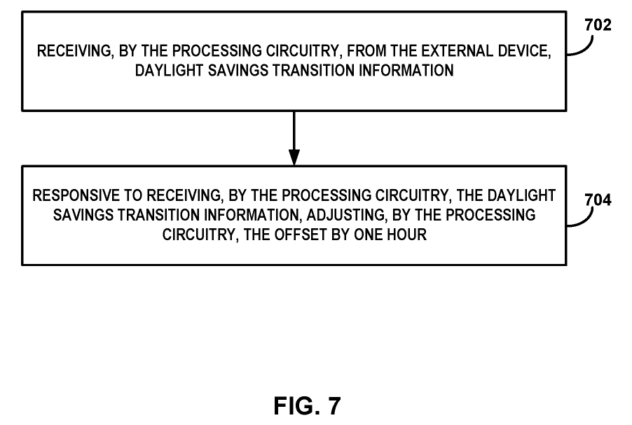

Examples of techniques for updating offset 44 based on information received from an external device (e.g., programmer 204) to account for daylight savings transitions and time zone changes (e.g., due to travel) are described in relation to FIGS. 7 through 9.

FIG. 7 is a flow chart illustrating an example technique for updating an offset to time information for an IMD. The example technique of FIG. 7 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 7.

In some examples, after time information 42 and offset 44 are set using a technique such as the technique described in relation to FIG. 5, offset 44 may be updated to adjust for daylight savings transitions based on information exchanged between IMD 210 and an external device (e.g., as described in relation to FIG. 7).

In the example of FIG. 7, processing circuitry 30 receives, from an external device (e.g., programmer 204), daylight savings transition information (702). For example, programmer 204, via processing circuitry 82 and communication circuitry 88, may transmit daylight savings transition information (e.g., a one-hour offset adjustment or an updated offset value that reflects a one-hour adjustment) after determining, via processing circuitry 82, that a daylight savings transition has occurred. In some examples, processing circuitry 82 is configured to determine whether a daylight transition has occurred based on time information 42 and offset 44 received from IMD 210. In other examples, processing circuitry 82 may be configured to transmit, via communication circuitry 88, daylight savings transition information based on a user input indication or otherwise received or determined indication that a daylight savings transition has occurred.

Responsive to receiving daylight savings transition information from the external device, processing circuitry 30 adjusts offset 44 by one hour (704). In some examples, processing circuitry 30 is configured to apply to offset 44 a one-hour offset adjustment received from the external device, via telemetry circuitry 36. In other examples, processing circuitry 30 is configured to replace the stored value of offset 44 with an updated offset value received from the external device, via telemetry circuitry 36, that reflects a one-hour adjustment.

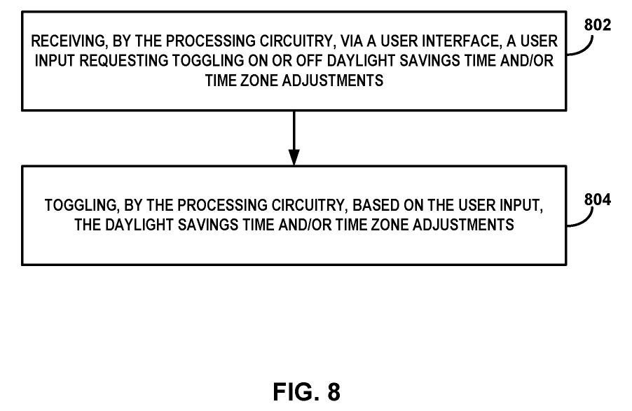

In some examples, daylight savings time adjustments can be turned off based on user preference or for jurisdictions that do not observe daylight savings time. FIG. 8 is a flow chart illustrating an example technique for toggling on or off daylight savings time adjustments for an IMD. The example technique of FIG. 8 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 8.

In the example of FIG. 8, processing circuitry 30 receives, via a user interface, a user input requesting toggling on or off daylight savings time adjustments or time zone adjustments, or both (802). For example, a user input to toggle on/off daylight savings time and/or time zone adjustments may be entered via user interface 86 of programmer 204. Responsive to the user input, processing circuitry 82 may be configured to transmit, via communication circuitry 88, an instruction to IMD 210 that causes processing circuitry 30 to toggle on/off daylight savings time and/or time zone adjustments. In this manner, processing circuitry 30 toggles on/off the daylight savings time and/or time zone adjustments based on the user input (804). Alternatively, processing circuitry 30 may toggle on/off daylight savings time and/or time zone adjustments based on a user input received via another user interface (e.g., a user interface of IMD 210 itself, or the user interface of another external device).

In other examples, toggling on/off daylight savings time and/or time zone adjustments is performed entirely by processing circuitry 82 of programmer 204. For example, programmer 204 may discontinue transmission of daylight savings time and/or time zone adjustments to IMD 210 after the daylight savings time and/or time zone adjustments are toggled off. Prior to discontinuing transmission of daylight savings time adjustments, programmer 204 may adjust the offset to standard time (if not already the case).

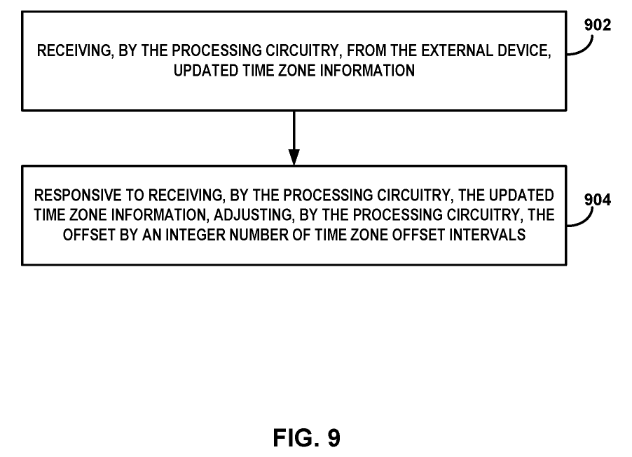

FIG. 9 is a flow chart illustrating an example technique for updating an offset to time information for an IMD. The example technique of FIG. 9 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 9.

In some examples, after time information 42 and offset 44 are set using a technique such as the technique described in relation to FIG. 5, offset 44 may be updated to adjust for a change of time zone (e.g., due to travel) based on information exchanged between IMD 210 and an external device (e.g., as described in relation to FIG. 9).

In the example of FIG. 9, processing circuitry 30 receives, from an external device (e.g., programmer 204), updated time zone information (902). For example, processing circuitry 30 may receive, via telemetry circuitry 36, updated time zone information (e.g., an N-hour offset adjustment or an updated offset value that reflects an N-hour adjustment, where N is an integer value) from programmer 204. In some examples, processing circuitry 82 is configured to transmit the updated time zone information, via communication circuitry 88, after determining that a difference of N hours exists between a user input time zone and a previous time zone of IMD 210 based on a comparison between the user input time zone and time zone information (e.g., predetermined time zone, or time zone derived from time information 42 and offset 44) received from IMD 210. In other examples, processing circuitry 82 may transmit, via communication circuitry 88, the updated time zone information based on a user input location or otherwise received or detected location information (e.g., WiFi or GPS based location information) from which an applicable time zone can be derived.

Responsive to receiving the updated time zone information, processing circuitry 30 adjusts offset 44 by an integer number of time zone offset intervals, such as hours or fractions thereof (904). In some examples, processing circuitry 30 is configured to apply to offset 44 an N-hour offset adjustment received from the external device, via telemetry circuitry 36. In other examples, processing circuitry 30 is configured to replace the stored value of offset 44 with an updated offset value received from the external device, via telemetry circuitry 36, that reflects an N-hour adjustment.

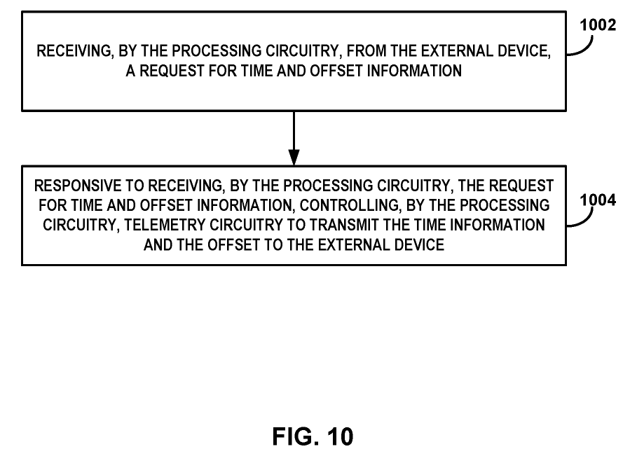

FIG. 10 is a flow chart illustrating an example technique for exchanging information between an IMD and an external device (e.g., programmer). The example technique of FIG. 10 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 10.

The example technique of FIG. 10 is a manner by which IMD 210 may be interrogated by an external device (e.g., programmer 204). In the example of FIG. 10, processing circuitry 30 receives, from an external device (e.g., programmer 204), a request for time and offset information (1002). For example, processing circuitry 30 may receive the request via telemetry circuitry 36 of IMD 210. Responsive to receiving the request for the time and offset information, processing circuitry 30 controls telemetry circuitry 36 to transmit time information 42 and offset 44 to the external device (1004). For example, processing circuitry 30 may be configured to send time information 42 and offset 44 as separate values or as a combined value (e.g., transmitting, to the external device, the current time according to IMD 210).

In some examples, the technique described in relation to FIG. 10 is implemented by IMD 210 to perform step 504 of FIG. 5, where IMD 210 may transmit time information 42 and/or offset 44 to an external device (e.g., programmer) in response to receiving a request for time and/or offset information from the external device. In other examples, IMD 210 may transmit time information 42 and/or offset 44 to an external device automatically (e.g., periodically or according to a schedule), without being interrogated by the external device.

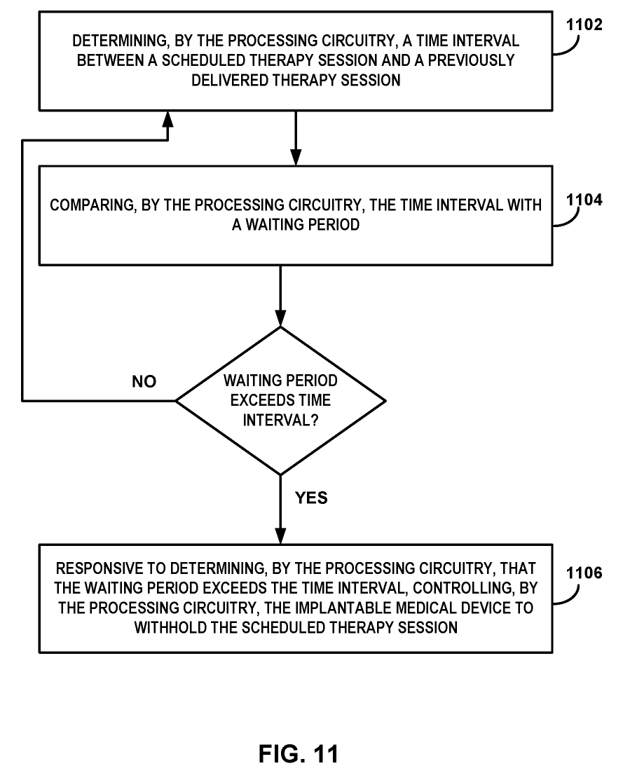

FIG. 11 is a flow chart illustrating an example technique for controlling an IMD to deliver therapy according to a therapy program, wherein therapy maybe temporarily withheld to safeguard against therapy occurring at a higher frequency than intended (e.g., due to large time zone changes). The example technique of FIG. 11 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210, the steps may also be performed by another device, alone or in combination with IMD 210 and programmer 204. For example, one or more of processing circuitry 82 of programmer 204, primary processing circuitry 50 of external charging device 208, processing circuitry 30 of IMD 210, and/or remote (e.g., cloud-based) processing circuitry (such as server 112) may be configured to perform any of the steps discussed in relation to FIG. 11.

In the example of FIG. 11, processing circuitry 30 determines a time interval between a scheduled therapy session and a previously delivered therapy session (1102). For example, processing circuitry 30 may calculate the difference in time between an upcoming therapy session and a past (e.g., most recent) therapy session. Processing circuitry 30 then compares the time interval (i.e., the calculated difference) with a waiting period (e.g., 24 hours or any other specified waiting period) (1104). Responsive to determining that the waiting period exceeds the time interval, processing circuitry 30 may control IMD 210 to withhold the scheduled therapy session (1106). For example, processing circuitry 30 may skip the scheduled therapy session or shift the therapy schedule to account for the waiting period. A waiting period according to the example technique of FIG. 11 may prevent stimulation or other therapy from occurring at higher than scheduled frequency due to time zone changes or daylight savings transitions that would cause multiple occurrences of the same therapy session (e.g., one before and one after the daylight savings transition and/or time zone change). The waiting period may also prevent therapy sessions from occurring too close to one another because of large time zone changes (e.g., due to overseas travel to a much later time zone). Processing circuitry 30 may be configured to shift the therapy schedule in 24 hour increments (or other appropriate time increments) to maintain therapy delivery at the same times but only change the days.

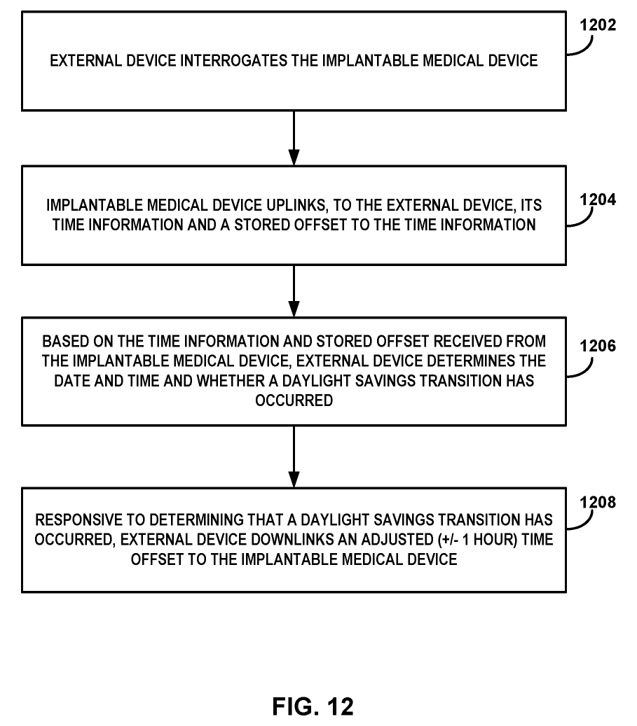

FIG. 12 is a flow chart illustrating an example technique for exchanging information between an IMD and an external device (e.g., programmer) to update an offset to time information for the IMD. The example technique of FIG. 12 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 12.

In the example of FIG. 12, an external device (e.g., programmer 204) interrogates IMD 210 (1202). For example, programmer 204, via processing circuitry 82 and communication circuitry 88, may transmit to IMD 210 a request for time and offset information. IMD 210, via processing circuitry 30 and telemetry circuitry 36, may responsively uplink to the external device (e.g., programmer 204) its time information (time information 42) and a stored offset (offset 44) to the time information (1204). Based on the time information and stored offset received from IMD 210, the external device (e.g., programmer 204, via processing circuitry 82), may determine the date and time and whether a daylight savings transition has occurred (1206). Responsive to determining that a daylight savings transition has occurred, the external device (e.g., programmer 204, via processing circuitry 82 and communication circuitry 88) may downlink a one-hour adjusted time offset that replaces offset 44 to IMD 210 (1208).

In some examples, if a daylight-saving transition has occurred software (SW) of programmer 204 will downlink a new local time offset (e.g., an adjustment or new offset value for offset 44) into firmware (FW) of IMD 210. FW of IMD 210 may reuse the same logic that was used to setup a therapy schedule with a given time zone (local time offset) and shift the schedule one hour ahead or back. In this way, the patient receives scheduled stimulation at the same time of the day, independent of daylight savings.

In some examples, daylight savings time adjustments can be “toggled off” or ignored, as previously described herein, for patients who prefer a sensory or “body clock” stimulation cadence or who move to time zone that does not observe daylight savings, and to guard against future daylight savings legislation.

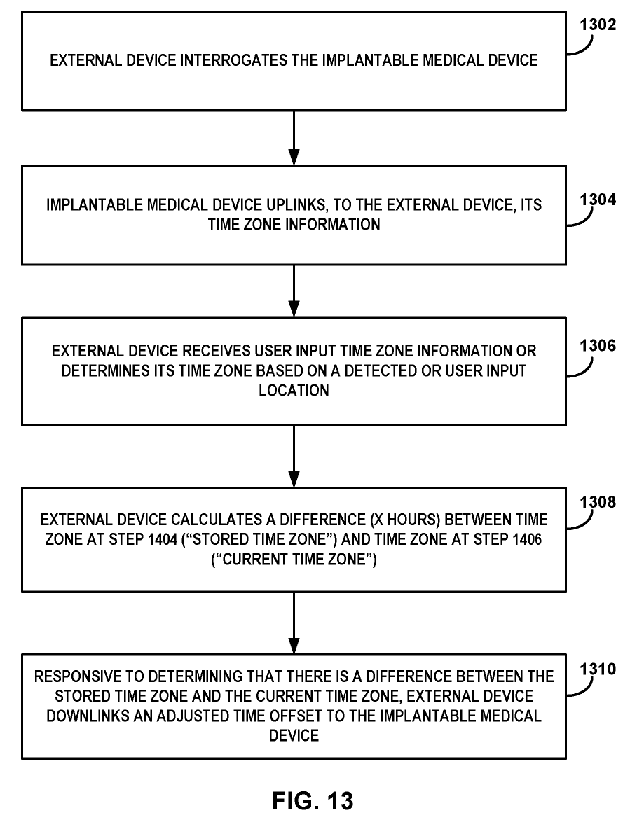

FIG. 13 is a flow chart illustrating an example technique for exchanging information between an IMD and an external device (e.g., programmer) to update an offset to time information for the IMD. The example technique of FIG. 13 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 13.

In the example of FIG. 13, an external device (e.g., programmer 204) interrogates IMD 210 (1302). For example, programmer 204, via processing circuitry 82 and communication circuitry 88, may transmit to IMD 210 a request for time zone information (e.g., stored time zone or location and/or time and offset information from which the time zone can be derived). IMD 210, via processing circuitry 30 and telemetry circuitry 36, may responsively uplink to the external device (e.g., programmer 204) its time zone information (1304). The external device (e.g., programmer 204, via processing circuitry 82 and user interface 86 or communication circuitry 88) may receive user input time information or determine a time zone of the external device based on a detected or user input location (1306). The external device (e.g., programmer 204, via processing circuitry 82) may calculate a time difference between a stored time zone of IMD 210 as determined at step 1304 and a current time zone as determined at step 1306 (1308). Responsive to determining that the time difference is a nonzero value, the external device (e.g., programmer 204, via processing circuitry 82 and communication circuitry 88) may downlink an adjusted time offset that replaces offset 44 to IMD 210 (1310), wherein the adjusted time offset is calculated by adding or subtracting the time difference to or from the previously stored value of offset 44.

In an example scenario, if a patient travels to a different time-zone their stimulation schedule may be that of the time zone in which the last clinical interaction occurred. According to techniques described herein, the patient may select their new time-zone in the patient programmer. Upon interrogation with IMD 210, the patient programmer can detect the differences in the two time zones and shift the stimulation schedule by the same amount. For example, when traveling from Plymouth, Massachusetts to Plymouth, California, if the patient inputs the new time zone to be Pacific Time, the patient programmer can shift the schedule back by 3 hours, so that the patient continues to receive stimulation at the same time of the day when they are in Plymouth, California.

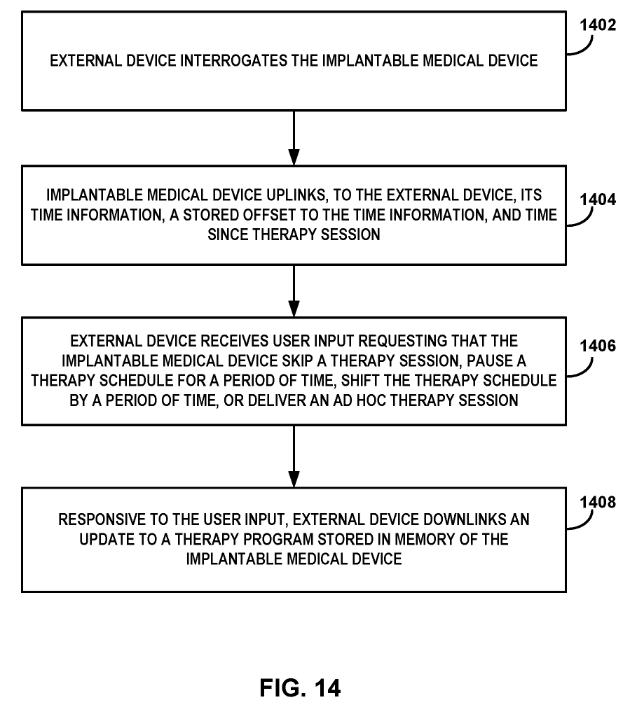

FIG. 14 is a flow chart illustrating an example technique for exchanging information between an IMD and an external device (e.g., programmer) to control the IMD to deliver therapy according to a therapy program and one or more user inputs. The example technique of FIG. 14 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 14.

In the example of FIG. 14, an external device (e.g., programmer 204) interrogates IMD 210 (1402). For example, programmer 204, via processing circuitry 82 and communication circuitry 88, may transmit to IMD 210 a request for time and offset information and time since one or more previous therapy sessions (e.g., time since most recent session). IMD 210, via processing circuitry 30 and telemetry circuitry 36, may responsively uplink to the external device (e.g., programmer 204) its time information 42, offset 44, and the time since one or more previous therapy sessions (1404). The external device (e.g., programmer 204, via processing circuitry 82 and user interface 86) may receive a user input requesting that IMD 210 skip a therapy session, pause a therapy schedule for a period of time (e.g., pause for 24 hours or any other specified period of time), shift the therapy schedule by a period of time (e.g., shift by 24 hours or any other specified period of time), or deliver an ad hoc therapy session (e.g., a user requested therapy session in addition to a therapy program’s scheduled therapy sessions) (1406). Responsive to the user input, the external device (e.g., programmer 204, via processing circuitry 82 and communication circuitry 88) may downlink an update to a therapy program stored in memory 32 of IMD 210 (1408). In some examples, the update implements the user input request by modifying the therapy program stored in memory 32 of IMD 210 or by replacing stored therapy program with a modified version.

In example scenarios, a patient programmer may present information in terms of time since last stimulation session, or time to next stimulation sessions, and the patient may use this information to get an ad hoc stimulation (e.g., direct IMD 210 to give them stimulation after 3600 seconds/1hour), pause stimulation schedule after 72000 seconds and resume it after 108000 seconds, or skip a stimulation session that is happening after 5400 seconds/1.5 hours etc. Allowing a patient to control therapy by requesting ad hoc stimulation, pausing stimulation, or by skipping or shifting scheduled therapy sessions may allow the patient to regulate therapy based on their specific needs rather than relying on a fixed schedule. To avoid changes that would be harmful to the patient or reduce efficacy of therapy, the system may impose limits on the patient's ability to make changes. For example, the system may limit adjustments to the therapy schedule based on time (e.g., +/- X hours or less), a specified waiting period between therapy sessions, a minimum or maximum number of therapy sessions per time interval, a maximum number of ad hoc sessions or pauses, or any other predefined limitation. In some examples, limitations on the patient’s ability to control therapy are specified by a clinician programmer.

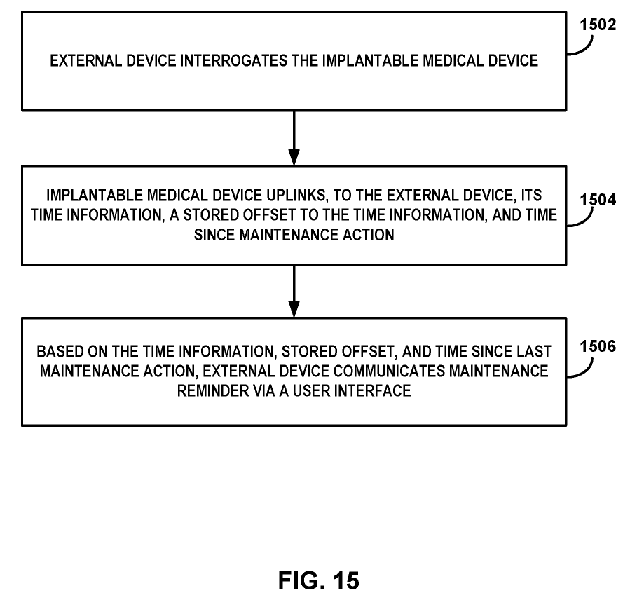

FIG. 15 is a flow chart illustrating an example technique for exchanging information between an IMD and an external device (e.g., programmer) to communicate maintenance reminders via a user interface of the external device. The example technique of FIG. 15 is discussed in relation to the components of IMD 210 as discussed in connection with FIG. 2, the components of programmer 204 as discussed in connection with FIG. 3, the components of external charging device 208 as discussed in connection with FIG. 4, but may be used with any of the devices of this disclosure (e.g., IMD 10, external charging device 108, programmer 104, and/or server 112 of FIG. 1). While the steps described herein are described as being performed by processing circuitry 30 of IMD 210 and processing circuitry 82 of programmer 204, the steps may also be performed by another device, alone or in combination with IMD 210 and programmer 204. For example, one or more of primary processing circuitry 50 of external charging device 208, processing circuitry 30 of IMD 210, and/or remote (e.g., cloud-based) processing circuitry (such as server 112) may be configured to perform any of the steps discussed in relation to FIG. 15.

In the example of FIG. 15, an external device (e.g., programmer 204) interrogates IMD 210 (1502). For example, programmer 204, via processing circuitry 82 and communication circuitry 88, may transmit to IMD 210 a request for time and offset information and time since one or more maintenance actions. In some examples, time since one or more maintenance actions may include, but is not limited to, time since most recent clinical visit (e.g., based on last time IMD 210 was interrogated by a clinician programmer), time since most recent recharging session (e.g., based on battery status or last time IMD 210 was connected to or interrogated by external charging device 208), time since external device 208 was last recharged. IMD 210, via processing circuitry 30 and telemetry circuitry 36, may responsively uplink to the external device (e.g., programmer 204) its time information 42, offset 44, and the time since one or more maintenance actions (1504). Based on the information uplinked from IMD 210 to the external device, the external device (e.g., programmer 204, via processing circuitry 82 and user interface 86) may communicate one or more maintenance reminders (1506). For example, processing circuitry 82 may control user interface 86 to provide a maintenance reminder in the form of a visual alert (e.g., text, calendar alert, banner, badge, icon, indicator light, or any other visual indication) or an audible alert (e.g., buzzer, ringtone, chime, spoken alert, or any other audible indication).

In an example scenario, a patient programmer may receive from IMD 210 information about the last clinician session, information about the current IMD time, and remind the patient about the amount of time passed since their last visit to the clinician’s office, and that it is time to schedule a visit. In another example scenario, a patient programmer may receive from IMD 210 information about past charging sessions (e.g., time since last recharge) and ask the patient to charge IMD 210, or charge their recharger and then charge IMD 210, etc. In another example scenario, a patient programmer may determine a stimulation session schedule and add information about the stimulation session schedule to a software App (e.g., to a calendar App) of the patient programmer, which may be a smartphone, tablet, wearable device, or any other type of personal electronic device. This may allow the patient to receive notifications regarding when stimulation sessions will occur.

Any of the techniques described in relation to FIGS. 6 through 15 may be fully or partially combined. Additionally, portions of techniques described in relation to FIGS. 6 through 15 may be removed, modified, replaced with functionally equivalent techniques, or combined with portions of other techniques described in relation to FIGS. 6 through 15.

This disclosure includes the following non-limiting examples.

Example 1: A system includes processing circuitry of an implantable medical device, the processing circuitry configured to: determine time information for the implantable medical device based on an internal clock of the implantable medical device and a reference time stored in a memory of the implantable medical device; control telemetry circuitry to transmit the time information to an external device; receive, from the external device, an offset to the time information; store the offset in the memory of the implantable medical device; and control the implantable medical device to deliver therapy according to the time information, the offset, and a therapy program that at least partially defines the therapy.

Example 2: The system of example 1, wherein the processing circuitry is configured to: determine a date based on the time information and the offset; compare the date with a daylight savings transition date; determine, based on the comparison, that the date exceeds the daylight savings transition date; and responsive to determining that the date exceeds the daylight savings transition date, adjust the offset by one hour.

Example 3: The system of example 1, wherein the processing circuitry is configured to: receive, from the external device, daylight savings transition information; and responsive to receiving the daylight savings transition information, adjust the offset by one hour.

Example 4: The system of any of examples 1 through 3, wherein the processing circuitry is configured to: receive, from the external device, updated time zone information; and responsive to receiving the updated time zone information, adjust the offset by an integer number of time zone offset intervals.

Example 5: The system of any of examples 1 through 4, wherein the processing circuitry is configured to: receive, via a user interface, a user input requesting toggling on or off at least one of daylight savings time adjustments or time zone adjustments, and toggle, based on the user input, the at least one of the daylight savings time adjustments or the time zone adjustments.

Example 6: The system of any of examples 1 through 5, wherein the processing circuitry is configured to: receive, from the external device, a request for time and offset information; and responsive to receiving the request for time and offset information, control telemetry circuitry to transmit the time information and the offset to the external device.

Example 7: The system of any of examples 1 through 6, wherein the processing circuitry is configured to retrieve, from memory of the implantable medical device, information of the therapy program for the implantable medical device, wherein the information includes therapy parameters and a therapy schedule defining delivery of therapy by the implantable medical device.

Example 8: The system of any of examples 1 through 7, wherein the processing circuitry is configured to: determine a time interval between a scheduled therapy session and a previously delivered therapy session; compare the time interval with a waiting period; determine, based on the comparison, that the waiting period exceeds the time interval; and responsive to determining that the waiting period exceeds the time interval, control the implantable medical device to withhold the scheduled therapy session.

Example 9: The system of any of examples 1 through 8, wherein the processing circuitry is configured to control the implantable medical device to deliver electrical stimulation therapy to one or more of a sacral nerve or tibial nerve according to the therapy program.

Example 10: The system of any of examples 1 through 9, further comprising the implantable medical device comprising the processing circuitry and the telemetry circuitry.

Example 11: A method including: determining, by processing circuitry, time information for an implantable medical device based on an internal clock of the implantable medical device and a reference time stored in a memory of the implantable medical device; controlling, by the processing circuitry, telemetry circuitry to transmit the time information to an external device; receiving, by the processing circuitry, from the external device, an offset to the time information; storing, by the processing circuitry, the offset in the memory of the implantable medical device; and controlling, by the processing circuitry, the implantable medical device to deliver therapy according to the time information, the offset, and a therapy program that at least partially defines the therapy.

Example 12: The method of example 11, further including: determining, by the processing circuitry, a date based on the internal clock and the reference time; comparing, by the processing circuitry, the date with a daylight savings transition date; determining, based on the comparison, that the date exceeds the daylight savings transition date; and responsive to determining, by the processing circuitry, that the date exceeds the daylight savings transition date, adjusting, by the processing circuitry, the offset by one hour.

Example 13: The method of example 11, further including: receiving, by the processing circuitry, from the external device, daylight savings transition information; and responsive to receiving, by the processing circuitry, the daylight savings transition information, adjusting, by the processing circuitry, the offset by one hour.

Example 14: The method of any of examples 11 through 13, further including: receiving, by the processing circuitry, from the external device, updated time zone information; and responsive to receiving, by the processing circuitry, the updated time zone information, adjusting, by the processing circuitry, the offset by an integer number of time zone offset intervals.

Example 15: The method of any of examples 11 through 14, further comprising: receiving, by the processing circuitry, via a user interface, a user input requesting toggling on or off at least one of daylight savings time adjustments or time zone adjustments, and toggling, by the processing circuitry, based on the user input, the at least one of the daylight savings time adjustments or the time zone adjustments.

Example 16: The method of any of examples 11 through 15, further including: receiving, by the processing circuitry, from the external device, a request for time and offset information; and responsive to receiving, by the processing circuitry, the request for time and offset information, controlling, by the processing circuitry, telemetry circuitry to transmit the time information and the offset to the external device.

Example 17: The method of any of examples 11 through 16, further including retrieving, by the processing circuitry, from memory of the implantable medical device, information of the therapy program for the implantable medical device, wherein the information includes therapy parameters and a therapy schedule defining delivery of therapy by the implantable medical device.

Example 18: The method of any of examples 11 through 17, further including: determining, by the processing circuitry, a time interval between a scheduled therapy session and a previously delivered therapy session; comparing, by the processing circuitry, the time interval with a waiting period; determining, based on the comparison, that the waiting period exceeds the time interval; and responsive to determining, by the processing circuitry, that the waiting period exceeds the time interval, controlling, by the processing circuitry, the implantable medical device to withhold the scheduled therapy session.

Example 19: The method of any of examples 11 through 18, wherein the implantable medical device is controlled, by the processing circuitry, to deliver electrical stimulation therapy to one or more of a sacral nerve or tibial nerve according to the therapy program.

Example 20: A system including an external device and an implantable medical device, the implantable medical device including processing circuitry configured to: determine time information for the implantable medical device based on an internal clock of the implantable medical device and a reference time stored in a memory of the implantable medical device; control telemetry circuitry to transmit the time information and the offset to the external device; receive, from the external device, an offset to the time information; store the offset in the memory of the implantable medical device; and control the implantable medical device to deliver therapy according to the time information, the offset, and a therapy program that at least partially defines the therapy.

The techniques described in this disclosure may be implemented, at least in part, in hardware, software, firmware or any combination thereof. For example, various aspects of the described techniques may be implemented within one or more processors, such as fixed function processing circuitry and/or programmable processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

What is claimed is:

1. A system comprising:
processing circuitry of an implantable medical device, the processing circuitry configured to:
determine time information for the implantable medical device based on an internal clock of the implantable medical device and a reference time stored in a memory of the implantable medical device;
control telemetry circuitry to transmit the time information to an external device;
receive, from the external device, an offset to the time information;
store the offset in the memory of the implantable medical device; and
control the implantable medical device to deliver therapy according to the time information, the offset, and a therapy program that at least partially defines the therapy.

2. The system of claim 1, wherein the processing circuitry is configured to:
determine a date based on the time information and the offset;
compare the date with a daylight savings transition date;
determine, based on the comparison, that the date exceeds the daylight savings transition date; and
responsive to determining that the date exceeds the daylight savings transition date, adjust the offset by one hour.

3. The system of claim 1, wherein the processing circuitry is configured to:
receive, from the external device, daylight savings transition information; and
responsive to receiving the daylight savings transition information, adjust the offset by one hour.

4. The system of claim 1, wherein the processing circuitry is configured to:
receive, from the external device, updated time zone information; and
responsive to receiving the updated time zone information, adjust the offset by an integer number of time zone offset intervals.

5. The system of claim 1, wherein the processing circuitry is configured to:
receive, via a user interface, a user input requesting toggling on or off at least one of daylight savings time adjustments or time zone adjustments, and
toggle, based on the user input, the at least one of the daylight savings time adjustments or the time zone adjustments.

6. The system of claim 1, wherein the processing circuitry is configured to:
receive, from the external device, a request for time and offset information; and
responsive to receiving the request for time and offset information, control telemetry circuitry to transmit the time information and the offset to the external device.

7. The system of claim 1, wherein the processing circuitry is configured to retrieve, from memory of the implantable medical device, information of the therapy program for the implantable medical device, wherein the information includes therapy parameters and a therapy schedule defining delivery of therapy by the implantable medical device.

8. The system of claim 1, wherein the processing circuitry is configured to:
determine a time interval between a scheduled therapy session and a previously delivered therapy session;
compare the time interval with a waiting period;
determine, based on the comparison, that the waiting period exceeds the time interval; and
responsive to determining that the waiting period exceeds the time interval, control the implantable medical device to withhold the scheduled therapy session.

9. The system of claim 1, wherein the processing circuitry is configured to control the implantable medical device to deliver electrical stimulation therapy to one or more of a sacral nerve or tibial nerve according to the therapy program.

10. The system of claim 1, further comprising the implantable medical device comprising the processing circuitry and the telemetry circuitry.

11. A method comprising:
determining, by processing circuitry, time information for an implantable medical device based on an internal clock of the implantable medical device and a reference time stored in a memory of the implantable medical device;
controlling, by the processing circuitry, telemetry circuitry to transmit the time information to an external device;
receiving, by the processing circuitry, from the external device, an offset to the time information;
storing, by the processing circuitry, the offset in the memory of the implantable medical device; and
controlling, by the processing circuitry, the implantable medical device to deliver therapy according to the time information, the offset, and a therapy program that at least partially defines the therapy.

12. The method of claim 11, further comprising:
determining, by the processing circuitry, a date based on the internal clock and the reference time;
comparing, by the processing circuitry, the date with a daylight savings transition date;
determining, based on the comparison, that the date exceeds the daylight savings transition date; and
responsive to determining, by the processing circuitry, that the date exceeds the daylight savings transition date, adjusting, by the processing circuitry, the offset by one hour.

13. The method of claim 11, further comprising:
receiving, by the processing circuitry, from the external device, daylight savings transition information; and
responsive to receiving, by the processing circuitry, the daylight savings transition information, adjusting, by the processing circuitry, the offset by one hour.

14. The method of claim 11, further comprising:
receiving, by the processing circuitry, from the external device, updated time zone information; and
responsive to receiving, by the processing circuitry, the updated time zone information, adjusting, by the processing circuitry, the offset by an integer number of time zone offset intervals.

15. The method of claim 11, further comprising:
receiving, by the processing circuitry, via a user interface, a user input requesting toggling on or off at least one of daylight savings time adjustments or time zone adjustments, and
toggling, by the processing circuitry, based on the user input, the at least one of the daylight savings time adjustments or the time zone adjustments.

16. The method of claim 11, further comprising:
receiving, by the processing circuitry, from the external device, a request for time and offset information; and
responsive to receiving, by the processing circuitry, the request for time and offset information, controlling, by the processing circuitry, telemetry circuitry to transmit the time information and the offset to the external device.

17. The method of claim 11, further comprising:
retrieving, by the processing circuitry, from memory of the implantable medical device, information of the therapy program for the implantable medical device, wherein the information includes therapy parameters and a therapy schedule defining delivery of therapy by the implantable medical device.

18. The method of claim 11, further comprising:
determining, by the processing circuitry, a time interval between a scheduled therapy session and a previously delivered therapy session;
comparing, by the processing circuitry, the time interval with a waiting period;
determining, based on the comparison, that the waiting period exceeds the time interval; and
responsive to determining, by the processing circuitry, that the waiting period exceeds the time interval, controlling, by the processing circuitry, the implantable medical device to withhold the scheduled therapy session.

19. The method of claim 11, wherein the implantable medical device is controlled, by the processing circuitry, to deliver electrical stimulation therapy to one or more of a sacral nerve or tibial nerve according to the therapy program.

20. A system comprising:
an external device; and
an implantable medical device, the implantable medical device including processing circuitry configured to:
determine time information for the implantable medical device based on an internal clock of the implantable medical device and a reference time stored in a memory of the implantable medical device;
control telemetry circuitry to transmit the time information and the offset to the external device;
receive, from the external device, an offset to the time information;
store the offset in the memory of the implantable medical device; and
control the implantable medical device to deliver therapy according to the time information, the offset, and a therapy program that at least partially defines the therapy.
